(19) United States
(12) Patent Application Publication
(10) Pub. No.: US 2010/0137205 A1
(43) Pub. Date: Jun. 3, 2010

(54) Use of FGF-18 Protein, Target Proteins and Their Respective Encoding Nucleotide Sequences to Induce Cartilage Formation

(76) Inventor: Jeffrey Allen WHITSETT, Cincinnati, OH (US)

Correspondence Address: HASSE & NESBITT LLC, 8837 CHAPEL SQUARE DRIVE, SUITE C, CINCINNATI, OH, 45249, US

(21) Appl. No.: 12/463,849

(22) Filed: May 11, 2009

Related U.S. Application Data
(63) Continuation of application No. 10/551,105, filed on Sep. 26, 2005, now abandoned.
(60) Provisional application No. 60/458,224, filed on Mar. 27, 2003.

(51) Int. Cl. A61K 38/18; C07K 14/50; A61P 11/00; A61P 19/02; C12N 15/63; C12N 5/071

(52) U.S. Cl. 514/12; 530/399; 435/320.1; 435/325

(57) ABSTRACT

The use of fibroblast growth factor (FGF)-18 protein, certain of its downstream target genes and respective expressed proteins, in particular sonic hedgehog (Shh), Shh protein, β-catenin, β-catenin protein, and the Wnt family of proteins that stimulate β-catenin, and the respective nucleotide sequences encoding this protein, particularly for inducing cartilage formation, particularly for the purpose of generating, repairing, reconstructing, or de novo formation of, cartilaginous tissue. Therapies for which FGF-18 and the target proteins are useful include repair and reconstruction of various tissues in conducting airways such as the trachea, bronchi, lung and larynx caused by, for example, tracheal-bronchial abnormalities, tracheal-laryngo or bronchial malaria. Other therapies for which FGF-18 and the target proteins would be useful include other cartilaginous tissues, such as those of joint and skeletal tissue caused by, for example, arthritis and meniscus abnormalities in joints.

### TECHNICAL FIELD

The present application relates to fibroblast growth factor (FGF)-18 protein, the respective nucleotide sequences encoding this protein, certain of its downstream target genes and respective expressed proteins, in particular sonic hedgehog (Shh), Shh protein, β-catenin, β-catenin protein, and the Wnt family of proteins that stimulate β-catenin, and their use in inducing cartilage formation, particularly for the purpose of generating, repairing, reconstructing, or de novo formation of, cartilaginous tissue. The present application further relates to the use of compositions containing FGF-18 protein, and/or these target proteins for inducing such cartilage formation, or the respective gene encoding these proteins to induce such formation.

### BACKGROUND OF THE INVENTION

Cartilage is a specialized type of dense connective tissue consisting of cells embedded in a matrix. There are several kinds of cartilage. Translucent cartilage having a homogeneous matrix containing collagenous fibers is found in articular cartilage, in costal cartilages, in the septum of the nose, in the larynx and in the trachea. Articular cartilage is hyaline cartilage covering the articular surfaces of bones, while costal cartilage connects the true ribs and the sternum. Yellow cartilage is a network of elastic fibers holding cartilage cells which is primarily found in the epiglottis, the external ear, and the auditory tube. See U.S. Pat. No. 6,258,778 (Rogers et al), issued Jul. 10, 2001.

Cartilage tissue is made up of an extracellular matrix primarily comprised of collagens, glycosaminoglycans, and proteoglycans, along with chondrocyte cells, which are synthesized and secrete these components that assemble into cartilage. These components, and the water entrapped within these organic matrix elements, yield the unique elastic properties and strength of cartilage. Wozney et al, Science, (1988) 242:1528-1533; Sporn et al, J. Cell. Biol., (1987) 105:1039-1045. See also U.S. Pat. No. 6,258,778 (Rogers et al), issued Jul. 10, 2001 Morphogenesis of cartilage is of fundamental importance in that development of this tissue underlies and determines the form of much of the endoskeleton of vertebrates. Cartilage is the dominant skeletal material of early embryonic life, and becomes a permanent part of craniofacial, auricular, laryngeal, costal, and articular structures. Junctions between skeletal elements are almost entirely cartilaginous, and it is the precise morphogenesis of articular surfaces that ensures normal joint development and efficient joint function. See Tickle et al, Curr. Opin. Genet. Dev., (1995) 5:478-84; Johnson et al, Cell., (1997) 90:979-90.

Like skeletal cartilage, the cartilage structures associated with the respiratory system arise from primitive structures of embryonic cartilage. The trachea forms as an outpouching of the endoderm shortly after it fuses into the embryonic endodermal gut tube. The primitive larynx is derived from the second and fourth pharengeal arches, caudal to the tracheal outpouch. Endodermal epithelial cells of the outpouch and its primary and secondary branches invade the surrounding mesoderm, initiating a process known as branching morphogenesis. Interactions of the proximal endoderm with the surrounding mesoderm result in chondrogenesis, and formation of a series of cartilage ring structures ensues. These cartilaginous rings provide flexible support that contributes to maintenance of airways after birth. The signals inducing formation of cartilage are extinguished or suppressed in the distal endoderm, thus the developing lung parenchyma remains essentially devoid of cartilage. See Larsen, Human Embryology, (Churchill Livingstone, Inc., NY, 1993).

In general, disturbances in the control of cartilage morphogenesis can result in dysmorphogenesis of the skeleton, larynx, trachea, ear, nose, and other cartilaginous tissues, and such an event usually has deleterious effects. While some such types of dysmorphogenesis can be lethal, others can result in devastating deformities that are difficult or impossible to remedy with reconstructive surgery. The relatively poor vascularization of cartilage is also a serious obstacle in the healing process required for recovery from any type of plastic surgery. In addition, injury to cartilage can cause serious morbidity to this tissue. Since cartilage tissue is generally resistant to healing, the extended period of time required for healing frequently enables recurrent damage and compromises recovery from cartilage injuries. See Cartilage Vol. 1-3 (B. K. Hall, Ed.; Academic Press, NY, 1983).

Abnormalities of cartilage formation and development are major medical problems in several diseases Likewise, cartilage damage and degeneration are associated with many clinical conditions of serious consequence. These cartilage-related disorders account for nearly $2.5 billion in health care cost per year worldwide. Current therapies focus on reducing inflammation, pain management and increasing range of motion where joints are involved. However, therapies and treatments directed at inhibiting cartilage degeneration or, more importantly, promoting the formation, growth and development of cartilage remain elusive.

Articular cartilage has recently been demonstrated to have a spontaneous repair response in the case of full-thickness cartilage defects. See Shapiro et al, J. Bone Jt. Surg., (1993) 75-A(4):532-53. However, this repair response is limited in terms of form and function. By contrast, there have been no meaningful repair processes in partial-thickness lesions limited to the cartilage itself. See Hunziker et al, J. Bone Jt. Surg., (1996) 78-A:721-33. While many repair techniques have been proposed over the past four decades, none have successfully regenerated long-lasting hyaline cartilage tissue to replace damaged cartilage. See Buckwalter et al, J. Bone Jt. Surg., (1997) 79-A:612-32. In fact, most of the surgical interventions to repair damaged cartilage have been directed toward the treatment of clinical symptoms, such as pain relief and functional restoration of joint structures and articulating surfaces, rather than regeneration of hyaline cartilage. See Johnson et al. Arthroscopy, (1989) 2:54-69.

An initial surgical attempt to restore the normal articulating surface of joint cartilage has been made with the introduction of Pridie's resurfacing technique. This chondral repair technique utilizes the disruption of subchondral bone to induce bleeding from the bone marrow, thus promoting the regular wound-healing mechanism in the cartilage defect site. See Pridie, “Proceedings of the British Orthopaedic Association,” J. Bone Jt. Surg., (1959) 41B:618-19. Since Pridie's abrasion arthroplasty, several subchondral disruption techniques have been introduced in an attempt to improve the healing mechanisms of repaired tissue. These include subchondral drilling, arthroscopic abrasion, and microfracture techniques. See Mitchell et al, J. Bone Jt. Surg., (1976) 58A(2):230-33; Brown et al, J. Sports Med., (1974) 2:27-46; Bert et al, Rheum. Dis. ClinNAm., (1993) 19:725-39; Rodrigo et al, Am. J. Knee Surg., (1994) 7(3):109-16.

Several experimental animal studies on full-thickness cartilage repair have also revealed that subchondral breaching techniques create a fibrin clot formed from bleeding in the region of the cartilage defect. This clot is subsequently infiltrated by mesenchymal cells, and acts as a three-dimensional scaffold for migrating progenitor cells. See Shapiro et al, J. Bone h. Surg., (1993) 75-A(4):532-53. Within six weeks, these cells gradually differentiate and completely fill the defect region with a hyaline-like repair cartilage. However, this newly synthesized repair cartilage typically contains significantly less proteoglycan than normal hyaline cartilage. See Mitchell et al, J. Bone Jt. Surg., (1976) 58A(2):230-33. The repair tissue at this stage is also more cellular than the adjacent normal cartilage and shows no structural integration with the residual cartilage. As a result, there will be degeneration and fissuring in the cartilage tissue. While the full-thickness defect spontaneous repair mechanism is present for the first several weeks after surgical repair, it later fails due to inadequate mechanical and biochemical conditions in the repaired tissue. See O'Driscoll et al, J. Bone h. Surg., (1988) 70-A(4):595-606; Scherping et al, American Orthopaedic Society for Sports Medicine, (Toronto, ONT, Canada, 1995); Suh et al, Oper. Tech. Orthop., (1997) 7:270-78.

The concept of tissue engineering as an approach to cartilage repair was first proposed by Green in 1977. See Green et al, Clin. Orthop. Relat. Res., (1977) 124:237-50. In this approach, chondrocytes grown in an ex vivo environment are transplanted into the cartilage defect. Clinical application of such a tissue engineering approach was first attempted by a Swedish group. See Brittberg et al, New Engl. J. Med., (1994) 331(14):889-95. Chondral repair techniques utilizing laboratory-grown cells have attracted significant attention. See Buckwalter et al, J. Bone Jt. Surg., (1997) 79-A:612-32. Recently, tissue engineering concepts have been introduced to develop cell-based repair approaches for articular cartilage. See Freed et al, J. Biomed. Mater. Res., (1993) 27:11-23; Vacanti et al, AJSM, (1994) 22(4):485-88. Tissue engineering of articular cartilage involves the isolation of articular chondrocytes or their precursor cells that can be expanded in vitro and then seeded into a biocompatible matrix, or scaffold, for cultivation and subsequent implantation into the joint. The type of cell used to engineer cartilage is critical to the long-term outcome. Different cell populations that have been investigated in the experimental studies include matured articular chondrocytes, epiphyseal chondrocytes, mesenchymal stem cells, bone marrow stromal cells, and perichondrocytes. See Robinson et al, Methods in Cartilage Research, (London: Academic Press, 1990), pp. 327-30; Bently et al, Nature, (1971) 230:385-88; Itay et al, Clin. Orthop. Relat. Res., (1987) 220:284-301; Wakitani et al, J. Bone Jt. Surg., 1989) 71-B:74-80; Butnariu-Ephrat et al, Clin. Orthop. Relat. Res., (1996) 330:234-43; Martin et al, J. Orthop. Res., (199) 16:181-89; Wakitani et al, J. Bone Jt. Surg., (1994) 76-A(4):579-92; Chu et al, J. Biomed. Mater. Res., (1995) 29:1147-54.

The choice of biomaterial is also critical to the success of such tissue engineering approaches in cartilage repair. A variety of biomaterials, naturally occurring and synthetic, biodegradable and non-biodegradable, have been used as potential cell-carrier substances for cartilage repair. See Grande et al, J. Biomed. Mater. Res., (1997) 34:211-20. The naturally occurring biomaterials include various forms of types I and II collagen-based biomaterial in the form of scaffold matrices, gels, or collagen/alginate composite gels. See Speer et al, Clin. Orthop. Relat. Res., (1979) 144:326-35; Sams et al, OA. Cartilage, (1995) 3:47-59; Frenkel et al, J. Bone Jt. Surg., (1997) 79-B:831-36; Nehrer et al, J. Biomed. Mater. Res., (1997) 38:95-104; Kang et al, OA. Cartilage, (1997) 5:139-43; Kimura et al, Clin. Orthop. Relat. Res., (1984) 186:231-39; Qi et al, J. Orthop. Res., (1997) 15(4):483-90. The synthetic polymer-based biomaterials include polyglycolic acid (PGA) and poly-L-lactic acid (PLLA), or their composite mixture. In cartilage tissue engineering, PGA, PLLA, and PGA/PLLA copolymers have been studied for their efficacy as chondrocyte-delivering scaffolds in vitro and in vivo. See Freed et al, J. Biomed. Mater. Res. (1994) 28:891-99; Woo et al, Plastic Reconstr. Surg., (1994) 94(2):233-37; Athanasiou et al, Biomaterials, (1996) 17:93-102. Several investigators have also found that some non-biodegradable polymer substances, such as polytetrafluoroethylene, polyethylmethacrylate, and hydroxyapatite/Dacron composites, also facilitate the restoration of an articular surface. Reisses et al, Transactions of the Orthopaedics Research Society, (New Orleans, La., 1994); Messner et al, Biomaterials, (1993) 14:513-21. The ideal cell-carrier substance is one that most closely mimics the naturally occurring environment in the articular cartilage matrix.

It has been shown that cartilage-specific extracellular matrix components such as type II collagen and glycosaminoglycan (GAG) can play a critical role in regulating expression of the chondrocytic phenotype and in supporting chondrogenesis both in vitro and in vivo. See Kosher et al, Dev. Biol., (1973) 35(2):210-20; Kosher et al, Nature, (1975) 258:327-30. Otherwise, chondrocytes can undergo de-differentiation and produce an inferior fibrocartilaginous matrix rich in type I collagen. This inferior matrix can then lead to a failure to form hyaline cartilage. See von Der Mark et al, Nature, (1977) 267:531-32; Sokoloff et al, J. Rheumatol., (1974) 1:1-10. Thus, the criteria for the choice of biomaterial in cartilage tissue engineering include biological friendliness and biomechanical strength. These features can provide a biochemically and biomechanically appropriate environment necessary for engineered cells to regenerate a long-lasting hyaline cartilage in the defect site. See Langer et al, Transactions of the 44th ORS, (New Orleans, 1998).

Also crucial to the tissue engineering process is the identification of growth factors that induce chondrogenesis. Many growth factors have been found to play a role in regulating cartilage development, remodeling or repair. For instance, it has previously been shown that the endogenous growth factors TGF-beta and various BMP family members induce both new cartilage and bone formation. However, specific signals that induce the formation of cartilage have previously remained unknown.

Restoration of epithelial tissue, such as tracheal tissues, after tissue injury is a complex process, which includes several critical events, including deposition of the extracellular matrix, tissue remodeling, and angiogenesis. These events are coordinated with epithelial cell migration and proliferation to restore the epithelial and/or mucosal barrier (i.e., in epithelial tissues such as tracheal epithelium which secrete mucous). The coordination of these events is believed to involve the interaction between different classes of cells as well as between cells and their extracellular matrix. See U.S. Pat. No. 6,465,205 (Hicks), issued Oct. 15, 2002.

Present approaches to tracheal repair include resection and reanastomosing the injured airway, replacement of the damaged portion by synthetic material, and use of autologous tissue for reconstruction of the tracheal defect. See Letang et al., “Experimental Reconstruction of the Canine Trachea with a Free Revascularized Small Bowel Graft,” Ann. Thorac. Surg., (1990) 49:955-58; Mulliken et al, “The Limits of Tracheal Resection with Primary Anastomosis Further Anatomical Studies in Man,” J. Thorac. Cardiovasc. Surg., (1968) 55:418 (abstract); Neville et al., “Prosthetic Reconstruction of the Trachea and Carina,” J. Thorac. Cardiovasc. Surg., (1976) 72:525-36. Recently, tissue engineering approaches have been used in such repair, including forming an in vivo tracheal cartilaginous scaffolding by injecting dissociated chondrocytes into a preformed synthetic construct. See Hirano et al., “Hydroxylapatite for Laryngotracheal Framework Construction,” Ann. Otol. Rhinol. Laryngol., (1989) 98:713-17; Okumura et al., “Experimental Study of a New Tracheal Prosthesis Made from Collagen Grafted Mesh,” Trans. Am. Soc. Artif. Organs., (1991) 37:M317-19; Langer et al., “Tissue Engineering,” Science, (1993) 260:920-26.

However, there is still a need for therapies to generate cartilage and to treat various abnormalities or injuries that occur in various cartilaginous tissues, including of those of the joints, eye and nose and especially the cartilaginous tissues of various connecting airways such as the trachea, bronchi, lung and larynx. In particular, there is a need to develop effective cartilage generation, repair and reconstruction techniques for abnormalities or injuries that occur in such cartilaginous tissues.

### BRIEF DESCRIPTION OF THE INVENTION

The present invention relates to the discovery that fibroblast growth factor (FGF)-18 protein is capable of inducing cartilage formation, as well as the use of nucleotides sequence(s) encoding FGE-18 protein for various purposes, including generation, repair, reconstruction, de novo formation or other formation of a variety of cartilaginous tissues. In particular, selective regulation of FGF-18 can induce cartilage programming during development of tissues, including tracheal-bronchial cartilage tissue formation in the conducting airways. FGF-18 protein and its receptor and signaling pathways can be used to induce new cartilage formation or expand cartilage growth in various sites of the body, including the tracheal-bronchial rings of the conducting airways and larynx, as well as other sites where cartilage deposition would be therapeutic or beneficial such as the cornea, nose, ear, ribs, sternum, joints and bones. Therapies for which FGF-18 are useful include repair and reconstruction of various tissues in conducting airways such as the trachea, bronchi, lung and larynx caused by, for example, congenital or pathological tracheal-bronchial abnormalities. Other therapies for which FGF-18 would be useful include other cartilaginous tissues, such as those of joint and skeletal tissue caused by, for example, arthritis and meniscus abnormalities in joints.

It has been found that FGF-18 provides an appropriate induction signal to mesenchymal cells that results in chondrogenesis and subsequent formation of cartilage. For example, mice with ectopic expression of FGF-18 in pulmonary epithelial cells have reproducible ectopic cartilage formation and expansion of cartilage at normal bronchial sites in the lung. These cartilage cells have been demonstrated histologically by alcion blue staining in ectopic regions in the lung, and by immunohistochemical staining for collagen type II, a marker of early cartilage differentiation and expansion. By contrast, ectopic expression of FGF-7 or FGF-10 does not reproduce the changes in cartilage observed in mice with ectopic expression of FGF-18, thus demonstrating a unique role of FGF-18 signaling in the chondrogenetic process to induce new cartilage formation or expand cartilage growth.

The present invention further relates to the discovery that certain downstream target genes and their respective expressed proteins can induce or enhance cartilage formation, alone or in combination with FGF-18. These downstream target genes and respective expressed proteins are sonic hedgehog (Shh), Shh protein, β-catenin, β-catenin protein, and the Wnt family of proteins that stimulate β-catenin.

One embodiment of the present invention relates to a pharmaceutical composition comprising at least one cartilage formation inducing protein selected from the group consisting of FGF-18, Shh, β-catenin, and Wnt proteins, in an amount effective to induce cartilage formation.

Another embodiment of the present invention relates to a method for inducing cartilage formation in an affected area of a patient requiring such treatment comprising the step of administering to the affected area a pharmaceutical composition containing an amount of at least one cartilage formation inducing protein selected from the group consisting of FGF-18, Shh, β-catenin, and Wnt proteins effective to induce cartilage formation in the affected area.

Another embodiment of the present invention relates to an expression vector comprising at least one nucleotide sequence encoding at least one cartilage formation inducing protein selected from the group consisting of FGF-18, Shh, β-catenin, and Wnt proteins.

Another embodiment of the present invention relates to a method of expressing FGF-18 protein in a cell in vitro, comprising the step of providing an expression vector comprising at least one nucleotide sequences encoding at least one cartilage formation inducing protein selected from the group consisting of FGF-18, Shh, β-catenin, and Wnt proteins.

Another embodiment of the present invention relates to a method for treating a patient in need of cartilage regeneration, repair, reconstruction, de novo formation or other cartilage formation in an affected area of the patient, the method comprising the step of introducing to the affected area an expression vector comprising at least one nucleotide sequence encoding at least one cartilage formation inducing protein selected from the group consisting of FGF-18, Shh, β-catenin, and Wnt proteins to form cartilage in the cells.

Another embodiment of the present invention relates to a method for treating a patient in need of cartilage regeneration, repair, reconstruction, de novo formation or other cartilage formation in an affected area of the patient, the method comprising the step of introducing to the affected area at least one cartilage formation inducing protein selected from the group consisting of FGF-18, Shh, β-catenin, and Wnt proteins in an amount effective to induce formation of cartilage.

Another embodiment of the present invention relates to a cell culture comprising cells in a medium capable of sustaining cell growth, the cells having introduced therein an expression vector comprising at least one nucleotide sequence encoding at least one cartilage formation inducing protein selected from the group consisting of FGF-18, Shh, β-catenin, and Wnt proteins to form cartilage in the cells.

Another embodiment of the present invention relates to a cell culture comprising: (a) cells capable of producing cartilage in the presence of at least one cartilage formation inducing protein selected from the group consisting of FGF-18, Shh, β-catenin, and Wnt proteins; and (b) a medium capable of sustaining cell growth that contains an amount of the at least one cartilage formation inducing protein to induce formation of cartilage.

Another embodiment of the present invention relates to a method for preparing a cell culture for inducing cartilage formation in vitro in cells in a medium capable of sustaining cell growth, the method comprising the step of introducing into the cells an expression vector comprising at least one nucleotide sequence encoding at least one cartilage formation inducing protein selected from the group consisting of FGF-18, Shh, β-catenin, and Wnt proteins.

Another embodiment of the present invention relates to a cell culture comprising: (a) a first group of cells in a medium capable of sustaining cell growth; and (b) a second of group of cells of a type different from the first group of cells and co-cultured therewith, the second group of cells having introduced therein an expression vector comprising at least one nucleotide sequence encoding at least one cartilage formation inducing protein selected from the group consisting of FGF-18, Shh, β-catenin, and Wnt proteins to induce formation of cartilage.

Another embodiment of the present invention relates to a method for treating a patient in need of cartilage regeneration, repair, reconstruction, de novo formation or other cartilage formation in an affected area of the patient, the method comprising the step of administering to the affected area at least one cartilage formation inducing protein selected from the group consisting of FGF-18, Shh, β-catenin, and Wnt proteins in an amount effective to induce formation of cartilage in the affected area.

### BRIEF DESCRIPTION OF DRAWINGS

FIG. 1 is an image of a Northern Blot analysis of transgene specific mouse FGF-18, total FGF-18, and β-actin mRNAs assessed in fetal mouse lung at E16 and correlated with the presence and absence of the transgenes.

FIG. 2 is an image (original magnification ×4) of the expression of FGF-18 perturbing lung histology after hematoxylin-eosin staining of lung tissue from control (A and C) and double transgenic pups (B, D) at E16 (A, B) and E19, (C, D).

FIG. 3 is an image of the edge of fetal lung from FGF-18 treated and control littermates.

FIG. 4 is an image (original magnification ×10) of the effects of FGF-18 on TTF-1, proSP-C, SP-B, and CCSP after immunostaining for TTF-1, proSP-C and SP-B (taken at original magnification ×20), and CCSP performed on lungs of wild type (WT) pups (A, C, E, G, I) or double transgenic pups (B, D, F, H, J) expressing FGF-18.

FIG. 5 is an image (original magnification ×10) of lungs from FGF-18 expressing and control littermates (E19) immunostained for PECAM (A,B) and α smooth muscle actin (C, D, E, F).

FIG. 6 is an image of lung tissue from control (A) and FGF-18 expressing (B) littermates.

FIG. 7 is an image of lungs dissected from fetal mice and stained with alcian blue, and tissue digested with KOH prior to photography under a dissecting microscope, of affected (A) and control (B) mice treated with doxycycline from E6.

FIG. 8 is an image (original magnification ×4) of hematoxylin-eosin staining of cartilage rings in wild type (A) and FGF-18 expressing mice at E16 (B).

FIG. 9 is an image (original magnification ×4) of in situ hybridization performed with radiolabeled FGF-18 antisense (A,B) and sense probes (B,C) on sections of fetal mouse tissue from wild type mice on E18.5 (upper panels).

FIG. 10 is an illustration of the three linear maps of constructs (above vertical arrow) used to generate lung-specific recombination at the Shh exon 2 locus (below vertical arrow) following administration of doxycycline.

FIG. 11 is an image of a PCR Blot analysis on mRNA extracted from heart (lane A) and lungs (lane B) removed from Shh^{Δ/-} mice treated with doxycycline throughout gestation, and mRNA extracted from lungs of Shh^{flx/-} mice not treated with doxycycline (lane C).

FIG. 12 is an illustration of the linear maps of SP-C-rtTA (upper map) and (tetO)7CMV-rShh (lower map) transgene constructs.

FIG. 13 is an image showing, respectively, Shh^{flx/flx} (A), Shh^{Δ/-} (E), Shh^{-/-} (I) and Shh^{rescue} (M) mice, lungs thereof (B, F, J and N), hematoxylin and eosin (H&E)-stained lung sections thereof (C, G, K and O), and lung sections thereof (D, H, L and P) immunostained for the biologically active N-terminal fragment of sonic hedgehog protein (SHH-N) at E18.5 after treatment with doxycycline from E0.5-18.5.

FIG. 14 is an image showing, respectively, the gross pulmonary morphology (A and B), lung sections immunostained with SHH-N (C and D), lung sections immunostained with α-phosphohistone H3 (αPH3) (E and F) and lung sections in situ hybridized with radiolabeled riboprobe for the SHH receptor Patched-1 (Ptch1) mRNA of Shh^{flx/flx} and Shh^{Δ/-} mice at E13.5 after treatment with doxycycline from E0.5-13.5.

FIG. 15 is an image showing, respectively, Clara Cell Secretory Protein (CCSP) immunostaining in lung sections (A, E, I and M), pro-Surfactant Protein-C(SPC) immunostaining in lung sections (B, F, J and N), forkhead box transcription factor-J1 (FOXJ1) immunostaining in lung sections (C, G, K and O) and Calcitonin Gene-Related Peptide (CGRP) immunostaining in lung section (D, H, L and P) from Shh^{flx/flx}, Shh^{Δ/-}, Shh^{-/-} and Shh^{rescue} mice at E18.5.

FIG. 16 is an image showing, respectively, lung sections immunostained for α-Smooth Muscle Actin (α-SMA) (A, D, G and J), lung sections for Platelet Endothelial Cell Adhesion Molecule (PECAM) (B, E, H and K), and lung sections hybridized with radiolabeled riboprobe for Vascular Endothelial Growth Factor-A (VegfA) mRNA (C, F, I and L) from Shh^{flx/flx}, Shh^{Δ/-}, Shh^{-/-} and Shh^{rescue} mice at E18.5.

FIG. 17 is a bar graph of showing fold changes in lung gene expression in Shh^{Δ/-} vs. Shh^{flx/flx}, Shh^{flx/-} control littermates.

FIG. 18 is an image showing, respectively, lungs and trachea stained with Alcian blue (A, B, C and D) from Shh^{flx/flx}, Shh^{Δ/-}, Shh^{-/-} and Shh^{rescue} mice at E18.5.

FIG. 19 is an image showing, respectively, β-galactosidase staining in sections of peripheral lung and trachea from a Ptch1^{LacZ/+} mouse at E11.5 (A-C), at E13.5 (D-F) and at E15.5 (G-I).

FIG. 20 is a bar graph showing the number of days of doxcycline treatment for Shh^{Δ/-} (A) and Shh^{Δ/Δ} (B-F) mice, the results of which are shown in FIG. 21.

FIG. 21 is an image showing, respectively, lungs and trachea at E18.5 from a Shh^{Δ/-} mouse treated with doxycycline from E0.5-18.5 (A), from a Shh^{Δ/Δ} mouse treated with doxycycline from E0.5-18.5 (B), from a Shh^{Δ/Δ} mouse treated with doxycycline from E0.5-8.5 (C), from a Shh^{Δ/Δ} mouse treated with doxycycline from E8.5-18.5 (D), from a Shh^{Δ/Δ} mouse treated with doxycycline from E8.5-12.5 (E), and from a Shh^{Δ/Δ} mouse treated with doxycycline from E13.5-18.5 (F).

### BRIEF DESCRIPTION OF SEQUENCE LISTINGS

SEQ ID NO:1 shows the nucleotide sequence of the cDNA for FGF-18 (house mouse).

SEQ ID NO:2 shows the amino acid sequence for FGF-18 protein (house mouse).

SEQ ID NO:3 shows the nucleotide sequence of the cDNA for FGF-18 (human).

SEQ ID NO:4 shows the amino acid sequence for FGF-18 protein (human).

SEQ ID NO:5 shows the nucleotide sequence of the cDNA for Shh (house mouse).

SEQ ID NO:6 shows the amino acid sequence for Shh protein (house mouse).

SEQ ID NO:7 shows the nucleotide sequence of the cDNA for β-catenin (house mouse).

SEQ ID NO:8 shows the amino acid sequence for β-catenin protein (house mouse).

SEQ ID NO:9 shows the nucleotide sequence of the cDNA for Wnt1 (house mouse).

SEQ ID NO:10 shows the amino acid sequence for Wnt1 protein (house mouse).

SEQ ID NO:11 shows the nucleotide sequence of the cDNA for Wnt6 (house mouse).

SEQ ID NO:12 shows the amino acid sequence for Wnt6 protein (house mouse).

SEQ ID NO:13 shows the nucleotide sequence of the cDNA for Wnt7b (house mouse).

SEQ ID NO:14 shows the amino acid sequence for Wnt7b protein (house mouse).

### DETAILED DESCRIPTION OF THE INVENTION

1. Definitions

As used herein, the term “pluripotent” refers to the ability of a cell to differentiate into a wide variety of mature cell types. The term “multipotent” refers to the ability of a cell to differentiate into a more limited variety of mature cell types than that of a pluripotent cell.

As used herein, the term “stem cells” refers to any pluripotent cell type, and can be derived from embryonic or adult tissues.

As used herein, the term “autologous” refers to use of a recipient's or patient's own cells or tissues as a source for transplantation. By contrast, the term “heterologous” refers to cells or tissues transplanted from another human or species.

As used herein, the term “somatic” refers to cells other than those arising from germ cells, germ cells being the cells that produce gametes, i.e., spermatozoa and ova.

As used herein, the term “cartilaginous tissue” refers to chondrocytes, and tissue which is formed by chondrocytes, which demonstrate the histological and compositional characteristics of cartilage.

As used herein, the term “gene” means a sequence of genetic material (e.g., DNA and RNA) that carries the information encoding a polypeptide (e.g., protein).

Unless otherwise indicated herein, the term “polypeptide” means a protein, polypeptide or peptide.

As used herein, the term “vector” means an agent comprising, consisting essentially of, or consisting of a DNA or RNA molecule capable of introducing a nucleic acid sequence(s) into a cell, resulting in the expression of the nucleic acid sequence(s) in the cell. Examples include but are not limited to a modified plasmid or virus that carries a gene or cDNA into a suitable host cell and there directs expression or synthesis of the encoded polypeptide.

As used herein, the terms “FGF-18” and “FGF-18 protein” refer to Fibroblast Growth Factor-18, the polypeptide (FGF-18 protein) that is capable of inducing formation of cartilage. FGF-18 protein (house mouse) has the amino acid sequence shown in SEQ ID NO:2 and GenBank accession number AB004639, while FGF-18 protein (human) has the amino acid sequence shown in SEQ. ID NO:4 and GenBank accession number AB007422. See Ohbayashi et al, “Structure and Expression of the mRNA Encoding a Novel Fibroblast Growth Factor, FGF-18,” J. Biol. Chem., (1998) 273(29):18161-64, which is incorporated by reference. The amino acid sequence of SEQ ID NO:2 is identical to that of GenBank file AF075291, which includes untranslated flanking nucleotide sequences lying 5′ and 3′ to the coding sequences. See Hu et al., “FGF-18, A Novel Member of the Fibroblast Growth Factor Family, Stimulates Hepatic and Intestinal Proliferation,” Mol. Cell. Biol., (1998) 18(10):6063-74, which is incorporated by reference.

As used herein, the terms “nucleotide sequence(s) encoding FGF-18” or “nucleotide sequence(s) encoding FGF-18 protein” refer to the portion of the FGF-18 gene that codes for the polypeptide amino acid sequences of the bioactive FGF-18 protein. The cDNA of FGF-18 (house mouse) has the nucleotide sequence shown in SEQ ID NO:1 and has the GenBank accession number NM_{—}008005 and AF075291, while the cDNA of FGF-18 (human) has the nucleotide sequence shown in SEQ ID NO:3 and has the GenBank accession numbers NM_{—}033649, NM_{—}003862 and AF075292. See Ohbayashi et al, “Structure and Expression of the mRNA Encoding a Novel Fibroblast Growth Factor, FGF-18,” J. Biol. Chem., (1998) 273(29):18161-64, which is incorporated by reference. GenBank sequence file NM_{—}008005 comprises the coding sequence for FGF-18 that is identical to AB004639, as well as 5′ and 3′ flanking sequences. See Hu et al., “FGF-18, A Novel Member of the Fibroblast Growth Factor Family, Stimulates Hepatic and Intestinal Proliferation,” Mol. Cell. Biol., (1998) 18(10):6063-74, which is incorporated by reference.

As used herein, the terms “Shh” and “Shh protein” refer to the Sonic Hedgehog (Shh) protein, a polypeptide secreted by the respiratory epithelium that has been found to be necessary for normal tracheal-bronchial cartilage formation. Shh protein (house mouse) has the amino acid sequence shown in SEQ ID NO:6 and GenBank accession number NM_{—}009170.

As used herein, the terms “nucleotide sequence(s) encoding Shh” or “nucleotide sequence(s) encoding Shh protein” refer to the portion of the Shh gene that codes for the polypeptide amino acid sequences of the bioactive Shh protein. The cDNA of Shh (house mouse) has the nucleotide sequence shown in SEQ ID NO:5 and has the GenBank accession number NM_{—}009170.

As used herein, the terms “β-catenin” and “β-catenin protein” refer to the cellular protein that translocates to the nucleus of target cells, where it regulates gene expression in response to the secreted proteins of the Wnt family of polypeptides. β-catenin protein (house mouse) has the amino acid sequence shown in SEQ ID NO:8 and GenBank accession number NM_{—}007614.

As used herein, the terms “nucleotide sequence(s) encoding β-catenin” or “nucleotide sequence(s) encoding β-catenin protein” refer to the portion of the β-catenin gene that codes for the polypeptide amino acid sequences of the bioactive β-catenin protein. The cDNA of β-catenin (house mouse) has the nucleotide sequence shown in SEQ ID NO:7 and has the GenBank accession number NM_{—}007614.

As used herein, the terms “Wnt” and “Wnt protein” refer to a family of polypeptides that, via β-catenin expressed in respiratory epithelial cells, induce the production of signals in the respiratory epithelial cells that, in a reciprocal manner, induce cartilage formation and patterning in the underlying trachea-bronchial precursors. Representative examples of Wnt proteins include Wnt1, Wnt2, Wnt3a, Wnt5, Wnt6 and Wnt7b. Wnt1, Wnt6 and Wnt7b proteins (house mouse) have the amino acid sequence shown in, respectively, SEQ ID NO:10, NO:12 and NO:14, and the respective GenBank accession numbers NM_{—}021279, NM_{—}009526 and NM_{—}009528.

As used herein, the terms “nucleotide sequence(s) encoding Wnt” or “nucleotide sequence(s) encoding Wnt protein” refer to the portion of the Wnt gene that codes for the polypeptide amino acid sequences of the bioactive Wnt proteins. The cDNAs of Wnt1, Wnt6 and Wnt7b (house mouse) have the nucleotide sequences shown in, respectively, SEQ ID NO:9, NO:11 and NO:13 and the respective GenBank accession numbers NM_{—}021279, NM_{—}009526 and NM_{—}009528.

As used herein, the term “gene promoter” refers to that portion of the nucleotide sequence of the gene that regulates, controls or otherwise modulates (e.g., stimulates or suppresses) the expression by the particular gene. For example, a gene promoter can enhance transcription and/or translation of the gene, thus increasing the mRNA levels transcribed from that gene.

As used herein, the term “regulatory elements” refer to sequences required for processing of mRNA transcribed from a transgene or other mammalian gene. Such elements are well known to those with ordinary skill in the art, and include but are not limited to enhancers, introns, and poly-A sequences.

As used herein, the term “gene expression” refers to steps at the level of the DNA molecule that lead to the production of a gene polypeptide product. Thus, gene expression is to be understood herein as culminating in the production of the polypeptide encoded by the gene so expressed.

As used herein, the term “mammal” refers to humans and nonhuman mammals, including primates (e.g., humans, monkeys, baboons, macaques), dogs, cats, rabbits, rats, gerbils, hamsters, mice, horses, cows, goats, and other species commonly known as mammals.

As used herein, the term “intended recipient” is intended to include patients or individuals in need of therapeutic restoration or generation of cartilage or cartilaginous tissue(s).

As used herein, the term “affected area of the patient” means the area of the patient (e.g., joint, nose, ear, eye, trachea, etc.) requiring treatment to induce cartilage formation, or the area proximate thereto that is capable of inducing cartilage formation if treated according to the present invention.

As used herein, the term “comprising” means various agents, compositions, compounds, genes, polypeptides, components, steps and the like can be conjointly employed in the present invention. Accordingly, the term “comprising” encompasses the more restrictive terms “consisting essentially of and “consisting of.”

As used herein, the terms “therapeutic agent,” “pharmaceutical,” and “drug” are used interchangeably to refer to a pharmacological composition, formulation or compound, including those useful for administration to cells or tissues in vitro or in vivo to induce cartilage formation.

As used herein, the term “pharmaceutically acceptable salt” means non-toxic salts of compounds (which are generally prepared by reacting the free acid with a suitable organic or inorganic base) and include, but are not limited to, the acetate, benzenesulfonate, benzoate, bicarbonate, bisulfate, bitartrate, borate, bromide, calcium, camsylate, carbonate, chloride, clavulanate, citrate, dihydrochloride, edetate, edisylate, estolate, esylate, fumarate, gluceptate, gluconate, glutamate, glycollylarsanilate, hexylresorcinate, hydrabamine, hydrobromide, hydrochloride, hydroxynapthoate, iodide, isothionate, lactate, lactobionate, laurate, malate, maleate, mandlate, mesylate, methylbromide, methylnitrate, methylsulfate, mucate, napsylate, nitrate, oleate, oxalate, pamaote, palmitate, panthothenate, phosphate, diphospate, polygalacturonate, salicylate, stearate, subacetate, succinate, tannate, tartrate, teoclate, tosylate, triethiodide, and valerate salts, as well as mixtures of these salts.

As used herein, the term “E0.5” refers to embryonic day 0.5 of gestation. Similarly, the term “E18.5” refers to embryonic day 18.5 of gestation, and all intervening timepoints during embryonic development are referenced in the same manner.

As used herein, the term “immunostained” refers to a technique in which proteins are visualized within cells of a tissue section using labeled antibodies that bind to the protein of interest.

The following abbreviations are used herein for the following gene names. SHH-N (biologically active N-terminal fragment of sonic hedgehog), CCSP (Clara Cell secretory protein), Vegf4 (Vascular endothelial growth factor-4), PECAM (platelet endothelial cell adhesion molecule), CRE (Cre recombinase), FOXJ1 (forkhead box transcription factor-J1), α-SMA (alpha-smooth muscle myosin), TTF1 (Thyroid transcription factor-1), CGRP (Calcitonin gene-related peptide), aPH3 (alpha-phosphohistone H3), Hhip (Hedgehog interacting protein 1), Ptch (Patched-1), Tnc (tenascin C), Myh11 (myosin heavy chain 11), Srfpc (serum response factor co-factor protein), Cnn1 (calponin 1), Gli (GLI-Kruppel family member Gli 1), and Trpc2 (myosin light polypeptide 9).

All amounts, parts, ratios and percentages used herein are by weight unless otherwise specified.

### 2. Methods for Inducing or Stimulating Cartilage Formation

One aspect of the present invention are methods for inducing or stimulating cartilage formation through the use of at least one cartilage formation inducing protein selected from the group consisting of FGF-18, Shh, β-catenin, and Wnt proteins, and particularly the FGF-18 protein, alone or in combination with at least one target gene protein selected from the group consisting of the Shh, β-catenin, and Wnt proteins for enhancing the inducement or stimulation of cartilage formation. In these methods, the cartilage formation inducing protein (e.g., FGF-18) can be administered in vivo to induce or promote the formation of therapeutically useful cartilage-producing cells and/or cartilage in a patient requiring such therapy, can be administered to cells or tissues for in vitro induction of such cartilage formation with subsequent introduction (e.g., by implanting, transplanting, or other transfer method) of the cartilage or cartilage producing cells formed into the affected area (e.g., joint, nose, ear, eye, trachea, etc.) of the patient requiring such therapy, or can be administered in vitro to cells or tissues capable of inducing such cartilage formation with subsequent introduction (e.g., by implanting, transplanting, or other transfer method) of the induced cells or tissues in situ into the affected area (e.g., joint, nose, ear, eye, trachea, etc.) of patients requiring such therapy for subsequent formation of the inducted cartilage.

In carrying out the various embodiments of this method, the cartilage formation inducing protein is administered to cells capable of committing to a chondrocyte cell fate. Suitable cells of this type include but are not limited to immature or mature chondrocytes, mesenchymal cells, adult stem cells such as neural stem cells and bone marrow stromal cells, embryonic stem cells, fibroblasts, myoblasts, osteoblasts, and other pluripotent or multipotent cell types. Other somatic cells that can be induced to de-differentiate to a multipotent cell type can also be used. Administration of the cartilage formation inducing protein to these cell types promotes differentiation and maturation of the chondrocytic phenotype. The chondrocytes thus formed can synthesize and secrete collagenous matrix proteins to form cartilage structures in vitro or in vivo.

In one embodiment of this method, in vitro formation of cartilage is obtained by administration of the cartilage formation inducing protein to cultured immature or mature chondrocytes, mesenchymal cells, adult stem cells such as neural stem cells and bone marrow stromal cells, embryonic stem cells, fibroblasts, myoblasts, osteoblasts, and other pluripotent or multipotent cell types. Scaffold or matrix material can be used to promote formation of cartilage structures of the desired shapes or sizes. Natural scaffold or matrix can be provided by cartilage excised from donor sites in the intended recipient or patient, or from a heterologous donor source, including other human donors, or from animal sources, such as pig, dog, rodent, or other suitable mammalian species. Artificial sources for scaffold or matrix material include but are not limited to biodegradable and non-biodegradable synthetic polymer-based biomaterials such as polyglycolic acid (PGA) and poly-L-lactic acid (PLLA) or their composite mixtures, polytetrafluoroethylene, polyethylmethacrylate, or hydroxyapatite/Dacron composites. See Suh et al, “Application of Chitosan-based Polysaccharide Biomaterials in Cartilage Tissue Engineering: A Review,” Biomaterials, (2000) 21:2589-98; Freed et al, J. Biomed. Mater. Res., (1994) 28:891-99; Woo et al, Plastic. Reconstr. Surg., (1994) 94(2):233-37; Athanasiou et al, Biomaterials, (1996) 17:93-102, Reisses et al, Transactions of the Orthopaedics Research Society, (New Orleans, La., 1994); Messner et al, Biomaterials, (1993) 14:513-21, which are incorporated by reference. Scaffold or matrix material can be placed in the culture vessel containing cells that may be induced to differentiate into chondrocytes.

The cartilage formation inducing protein can be administered to the cells in vivo in solution added to the culture media, or can be impregnated in or otherwise bound to the scaffold or matrix material, or attached to beads or other particles that can be placed in the culture vessel, or in or on the scaffold or matrix material. Alternatively, the cartilage formation inducing protein can be produced by cells in culture, either through endogenous expression of the respective (e.g., FGF-18) gene, or by acquisition of the respective gene resulting from gene transfer to the cultured cells. Gene transfer can be achieved by introduction of a functional transgene comprising promoter and regulatory elements and the cartilage formation inducing protein encoding sequences contained within a viral vector, including but not limited to lentiviral, adenoviral, adeno-associated or other viral sequences capable of transducing mammalian cells. Gene transfer can also be mediated by liposomal or other carriers or mechanical delivery systems.

In another embodiment of this method, cells are grown in sheets rather than on a scaffold or matrix material. Similar modes of cartilage formation inducing protein administration can be provided to cultured immature or mature chondrocytes, mesenchymal cells, adult stem cells such as neural stem cells and bone marrow stromal cells, embryonic stem cells, fibroblasts, myoblasts, osteoblasts, and other pluripotent or multipotent cell types will promote differentiation of mature chondrocytes. The cells can grow and differentiate in confluent sheets of cartilage-producing cells. The sheets of differentiated cells thus produced can then be harvested and introduced to the affected area where cartilage formation is desired in the intended recipient. Administration of the cartilage formation inducing protein can also be included as part of a therapy to maintain the chondrocytic phenotype of the transplanted cells. Localized administration can be achieved using beads or other particles coated or impregnated with the cartilage formation inducing protein, or with the cartilage formation inducing protein covalently or otherwise functionally bound or linked to the beads or particles. These beads or particles can be made of biodegradable or non-biodegradable materials, including synthetic polymer-based biomaterials include PGA, PLLA or their composite mixtures, polytetrafluoroethylene, polyethylmethacrylate, or hydroxyapatite/Dacron composites. Non-corrosive metals, including but not limited to stainless steel, or precious metals, including but not limited to gold, can also be used as beads or other particles to which the cartilage formation inducing protein is covalently or otherwise bound or linked Metered administration of the cartilage formation inducing protein can also be directed by implantation of a mini-pump such that the outflow allows application of FGF-18 protein to the area where cartilage formation is desired.

Other embodiments of this method include in vivo formation of cartilage or cartilaginous tissues, either in situ in desired affected areas within the patient in need of such therapy or treatment, in ectopic sites within the intended recipient and subsequently introduced (e.g., transplanted) to the desired site, or in a human or non-human host/donor. In vivo formation of cartilage can be induced in situ where cartilage growth is desired by delivery of the cartilage formation inducing protein to cells capable of producing collagenous matrix materials that form cartilage. In cases where endogenous cells are capable of producing collagenous matrix materials, the cartilage formation inducing protein can be administered in the location where cartilage formation is desired. Localized administration can be achieved using beads or other particles coated or impregnated with the cartilage formation inducing protein, or with the cartilage formation inducing protein covalently or otherwise functionally bound or linked to the beads or particles, as previous described. In another embodiment where a scaffold or supporting matrix is desired to direct formation of cartilage structures, the cartilage formation inducing protein can be coated or impregnated on or in the scaffold or matrix, or covalently or otherwise bound or linked to the scaffold or matrix. Alternatively, the cartilage formation inducing protein can administered via expression of the respective gene acquired by gene transfer (as previously described) to endogenous cells surrounding the area of the scaffold or matrix implant.

In another embodiment of this method where endogenous cells are not capable of producing collagenous matrix materials, administration of the cartilage formation inducing protein can be accompanied by transplantation of immature or mature chondrocytes, mesenchymal cells, adult stem cells such as neural stem cells and bone marrow stromal cells, embryonic stem cells, fibroblasts, myoblasts, osteoblasts, or other pluripotent or multipotent cell types into the desired site of cartilage formation. These cells can be obtained from the intended recipient or patient as an autologous donation, or obtained from a heterologous donor source, including other human donors; cultured cells such as immature or mature chondrocytes, mesenchymal cells, adult stem cells such as neural stem cells and bone marrow stromal cells, embryonic stem cells, fibroblasts, myoblasts, osteoblasts, and other pluripotent or multipotent cell types; or from animal sources, such as pig, dog, rodent, or other suitable mammalian species.

In yet another embodiment of this method, the cartilage formation inducing protein can be administered via expression of the respective transgene acquired by cells through gene transfer, with gene transfer (as previously described) occurring prior to transplantation. Cells transduced with the cartilage formation inducing protein gene-containing vector are selectively cultivated and transplanted to the site where cartilage formation is desired. Suitable cells include any transducible non-transformed mammalian cell type, but primary cells harvested from the intended recipient or patient are preferred. Transducible cell types include, but are not limited to, cells such as fibroblast, osteoblasts, bone marrow stromal cells, neural stem cells, or myoblasts. Similar cell types from a heterologous donor can also be used.

When in vivo formation of cartilage cannot be induced in situ, or where de novo growth of cartilage is desired, cartilaginous tissues or structures can be grown in ectopic regions and transplanted to the desired site(s). Such ectopic regions can be located within the intended recipient or patient, and are most likely to be regions of endogenous cartilage. Examples of such regions include but are not limited to the cartilaginous regions of the sternum, ribs, pelvis, ears, and nose. Scaffold or matrix material can be implanted in suitable regions of naturally occurring cartilage, and the cartilage formation inducing protein is administered to recruit local chondrocytes to produce cartilage structures of the desired shape or size. Natural scaffold or matrix materials can be provided by cartilage excised from ectopic donor sites in the intended recipient or patient, or from a heterologous donor source, including other human donors, or from animal sources, such as pig, dog, rodent, or other suitable mammalian species. Artificial sources for scaffold or matrix material can include but are not limited to biodegradable and non-biodegradable synthetic polymer-based biomaterials previously described, with the cartilage formation inducing protein being coated on, impregnated within, or covalently or otherwise bound or linked to the scaffold or matrix material to induce formation of cartilage or cartilaginous structures of the desired size and shape, as previously described. Metered administration of the cartilage formation inducing protein can also be directed by implantation of a mini-pump, as previously described, or via expression of the respective gene acquired by gene transfer, as previously described. The cartilage or cartilaginous structures thus formed can subsequently be surgically excised and implanted or transplanted to the desired site in the intended recipient or patient. Alternatively, these cartilage or cartilaginous structures can be produced in a similar manner in a host/donor other than the intended recipient or patient, including other humans, or animals such as pig, dog, rodent, or other suitable mammalian species. The cartilage or cartilaginous structures thus formed can subsequently be surgically excised and transplanted to the desired site in the intended recipient or patient.

Embodiments of this method can use the cartilage formation inducing protein to induce cartilaginous tissue or other tissue formation in circumstances where such tissue is not normally formed, and has application in the healing of cartilage, for example articular cartilage tears, deformities and other cartilage defects in the affected area of patients (human or otherwise). The cartilage formation inducing protein can be employed for prophylactic use in preventing damage to cartilaginous tissue, as well as use in the improved fixation of cartilage to bone or other tissues, and in repairing defects to cartilage tissue. De novo cartilaginous tissue formation can be induced by the cartilage formation inducing protein for the repair of congenital, trauma induced, or other cartilage defects, and is also useful in surgery for attachment or repair of cartilage. The cartilage formation inducing protein can also be useful in the treatment of arthritis and other cartilage diseases, as well as other indications wherein it is desirable to heal or regenerate cartilage tissue. Such indications include, without limitation, regeneration or repair of injuries to the articular cartilage, e.g., cartilage of the joint such as the knee, ankle, shoulder or elbow.

In addition to articular cartilage, the cartilage formation inducing protein can be suitable for treatment of other translucent cartilages such as costal cartilages (e.g., cartilage connecting the ribs and sternum), in the septum of the nose, and particularly cartilage tissue formation relating to the conducting airways such as the trachea, bronchi, lung and larynx. For example, the cartilage formation inducing protein can be used to induce new cartilage formation or expand cartilage growth the tracheal-bronchial rings of the conducting airways, as well as the larynx. The cartilage formation inducing protein can also be useful in the treatment of problems in other affected areas of the patient such as the ear and the cornea.

The cartilage formation inducing protein can be used at any level or amount effective to stimulate or induce cartilage formation, for example, in the range of from about 0.1 ηg/ml to about 10 μg/ml.

### 3. Pharmaceutical Compositions and Packaged Drugs

The cartilage formation inducing protein (e.g., FGF-18) can be formulated as a pharmaceutical composition or packaged drug for use in the previously described methods. The pharmaceutical compositions include a therapeutically effective amount of the cartilage formation inducing protein, and optionally a pharmaceutically acceptable carrier. The packaged drug includes the cartilage formation inducing protein, optionally a pharmaceutically acceptable carrier, and instructions for administering or using the drug. The set of instructions can be written or printed on a sheet of paper, can be on the packaging associated with the packaged drug, can be in the form of electronic media or software (e.g., floppy disk or CD ROM disk) that can be loaded, installed (directly or by downloading from a remote site such as via a LAN, WAN or the Internet), or otherwise can be read by a computer, personal digital assistant (PDA) or other electronic device, or any other suitable method for providing instructions on how to administer the drug to treat the subject.

Although the cartilage formation inducing protein can be administered alone, it is preferably administered as part of a pharmaceutical formulation. Such formulations can include pharmaceutically acceptable carriers known to those skilled in the art, as well as other therapeutic agents. It will also be appreciated that the formulations of the present invention can be administered in various pharmaceutically acceptable forms, e.g., as pharmaceutically acceptable salts.

Appropriate dosages of the cartilage formation inducing protein administered in accordance with the present invention will depend on the desired location of formation of cartilage and the amount of cartilage needed, and can also vary from patient to patient. Determining an acceptable or optimal dosage will generally involve the balancing of the level of therapeutic benefit against any risk or deleterious side effects of the dose and treatment of the present invention. For a dose to be “therapeutically effective,” it must have the desired effect, i.e., induce the formation or expansion of cartilage or cartilaginous tissues in the desired location.

In addition to the cartilage formation inducing protein, pharmaceutical formulations of the present invention can also comprise additional compounds and/or compositions that will also aid in differentiation of chondrocytes and/or induction of cartilage formation. The ratio of the cartilage formation inducing protein to any additional compounds will depend upon the dose desired of each of the individual compounds. Preferably, the composition will be administered as a pharmaceutically-acceptable aqueous solution wherein the pharmaceutical formulation comprises: (1) from about 0.001% to about 10% cartilage formation inducing protein; (2) from about 10% to about 99% of a pharmaceutically-acceptable carrier; and (3) from about 0.001% to about 10% of any additional compound(s).

Administration of the cartilage formation inducing protein, with or without a pharmaceutically acceptable carrier(s) and/or additional compound(s), can be by any suitable route including coating on, impregnating within, or covalently or otherwise binding or linking to the scaffold or matrix material to induce formation of cartilage or cartilaginous structures of the desired size and shape. Metered administration of the cartilage formation inducing protein can also be directed by implantation of a mini-pump, as previously described, or via expression of the respective gene acquired by gene transfer, as previously described. Formulations suitable for minipump administration include aqueous and non-aqueous sterile injection solutions which can contain anti-oxidants, buffers, bacteriostats and solutes which render the formulation isotonic with the body fluids of the intended recipient; and aqueous and non-aqueous sterile suspensions which can include suspending agents and thickening agents. The formulations can be in unit or multi-dose containers, for example, sealed ampules and vials, and may be lyophilized, requiring only the addition of the sterile liquid carrier such as water for injections immediately prior to use. Extemporaneous injection solutions and suspensions can be prepared from sterile powders, granules and tablets of the kind previously described.

### 4. Enhancing Cartilage Growth and Patterning by Use of BMPs and TGFs

Another aspect of the present invention relates to the use of Bone Morphogenetic Proteins (“BMPs”) such as BMP-2 and BMP-4, Transforming Growth Factors (“TGFs”) such as TGF-β1, TGF-β2 and TGF-β3, or combinations thereof to enhance cartilage growth and patterning that is induced or stimulated by the cartilage formation inducing proteins, such as FGF-18. See U.S. Pat. No. 5,700,774 (Hattersley et al), issued Dec. 23, 1997 and U.S. Pat. No. 5,902,785 (Hattersley et al), issued May 11, 1999 (herein incorporated by reference), which disclose BMPs and TGFs suitable for use herein. Cartilage patterning and growth induced or stimulated by the cartilage formation inducing protein can be achieved by administering concurrent or separate doses of BMPs and/or TGFs at effective amounts, for example, in the range of from about 0.1 ηg/ml to about 10 μg/ml.

### EXAMPLES

The following example illustrates the capability of FGF-18 to induce cartilage formation in the conducting airways.

### A. Experimental Procedures

1. Transgenic Mice

A permanent transgenic mouse line bearing the SP-C-rtTA transgene is established in FVB/N background after oocyte injection of a plasmid construct consisting of 3.7 kb of the human SP-C promoter, placed 5′ to the rtTA gene construct. See Clark et al, Am. J. Physiol., (2001) 280:L705-15; Tichelaar et al, J. Biol. Chem., (2000) 275:11858-64; and Glasser et al, Am. J. Physiol., (1991) 261:L349-56. Mouse FGF-18 cDNA is inserted between the (teto)₇CMV promoter and the 3′ untranslated region of the bovine growth hormone gene. See Clark et al, Am. J. Physiol., (2001) 280:L705-15. The rtTA and (teto)₇ constructs are provided by Dr. Herman Bujard, ZMBH-Heidelberg. See Gossen et al, Proc. Natl. Acad. Sci. USA, (1992) 89:5547-51. Offspring of all founders are screened by Southern blot or PCR analysis. Mice transmitting the (teto)₇CMV-FGF-18 transgene are bred to SP-C-rtTA mice. The transgenic SP-C-rtTA “activator” line used is stable for more than three years in the vivarium. Heterozygous and homozygous (teto)₇CMV-FGF-18 mice are viable and without observable abnormalities. Two separate target lines bearing the (teto)₇CMV-FGF-18 transgene (lines A and B) are chosen for breeding to SP-C-rtTA activator mice. Transmission of both transgenes followed typical Mendelian inheritance patterns. All mice are maintained in a pathogen free vivarium. Doxycycline (0.5 mg/ml) is administered in drinking water or in the food pellets (25 mg/g; Harlen Teklar, Madison, Wis.) for the described time periods. The drinking solution containing doxycycline is changed 3 times per week, while activity of the doxycycline is stable in the food pellet. See Perl et al, Transgenic Res., (2002) 11:21-29.

2. RT-PCR

Tissues are homogenized in Tri-Zol (Life Technologies) and RNA is isolated according to the manufacturer's specifications. RNA is treated with DNAse prior to cDNA synthesis. Five μg RNA is reverse transcribed, then analyzed by PCR for murine FGF-18, and transgene specific FGF-18 and β-actin mRNAs. Transgene specific primers for mouse FGF-18 are designed to the (teto)₇CMV-FGF-18 transcript, and used for amplification. Primer A is located in the CMV minimal promoter (5′ to 3′) AGA CGC CAT CCA CGC TGT TTTG; primer B in the FGF-18 cDNA (5′ to 3′) CAG GAC TTG AAT GTG CTT CCC ACTG. FGF-18 mRNA is compared to that amplified for β-actin. FGF-18 mRNAs are also estimated using primers designed to amplify within the FGF-18 coding sequence using standard gel analysis of PCR products. FGF-18, FGF-10, SHH, BMP-4, and Sprouty-2 mRNAs are also determined by real time PCR of lung cDNA, after optimization of primers and conditions. Dams are placed on doxycycline throughout pregnancy, sacrificed on E15 and RNA extracted from the lungs of each pup. cDNA is prepared by reverse transcription and analyzed on the Smart Cycler® using primers to identify β-actin, FGF-18, BMP-4, Sprouty-2 and FGF-10. All results are normalized to β-actin.

3. Histology, Immunohistochemistry, and Electron Microscopy

To obtain fetal lung tissue, the fetuses are removed by hysterotomy after lethal injection of pentobarbital to the dam. The chest of fetal animals is opened and the tissue fixed with 4% paraformaldehyde at 4° C. Lungs from postnatal animals are inflation-fixed at 25 cm water pressure via a tracheal cannula with the same fixative. Tissue is fixed overnight, ished in PBS, dehydrated through a series of alcohols, and embedded in paraffin. Tissue sections are stained for SP-B, proSP-B, TTF-1, proSP-C, CCSP (Clara cell secretory protein), PECAM (peripheral endothelial cell adhesion molecules), α-smooth muscle actin, FOXJ1, and procollagen II using methods described in Clark et al, Am. J. Physiol., (2001) 280:L705-15 and Tichelaar et al, J. Biol. Chem., (2000) 275:11858-64. Cartilage is stained with Alcian blue and residual tissue dissolved in KOH prior to photography. For electron microscopy, tissue is fixed, prepared, and evaluated, as previously described. See Clark et al, Am. J. Physiol., (2001) 280:L705-15.

4. In Situ Hybridization

Expression of mouse FGF-18 mRNA is assessed by in situ hybridization using ³⁵S-labeled riboprobes for fetal and adult lungs, the latter after inflation fixation at 25 cm of water pressure. See Clark et al, Am. J. Physiol., (2001) 280:L705-15. Sense and antisense FGF-18 RNA probes are generated in PGEM32. Tissue is hybridized overnight at 50° C. Slides are coated with Kodak NTB2 emulsion, exposed for 7-14 days, and developed with Kodak D19. Whole mount in situ hybridization for mouse FGF-18, FGF-10, SHH, BMP-4, and Sprouty-2 are performed by digoxigenin labeled cDNA antisense and sense probes. Whole mount in situ hybridization is carried out on lungs of fetal day 12 embryos, whose dam had been on doxycycline throughout pregnancy. Anti-sense and sense probes are made from transcription vectors, using digoxigenin-UTP as label. After hybridization and ishing, anti-digoxigenin antibody coupled to alkaline phosphatase is adsorbed. The product is developed using BM purple alkaline phosphatase substrate.

5. RNA Analysis of Lung Tissue from (teto)₇CMV-FGF-18 Mice

Microarray hybridization and subsequent data analysis are performed using total lung RNA from (teto)₇CMV-FGF-18 mice and control littermates. Twenty-three RNAs are increased by >50% (2.2 to 31.42 fold) (see Table 1 below). Forty-seven RNAs are decreased significantly (see Table 2 below). These genes are components of the FGF-18 signaling pathway and are regulated by FGF-18 expression. As such, the genes in these lists, particularly those with increased expression, are genes that can regulate the production of cartilage. Mean and standard error are calculated from four independent hybridizations:

TABLE 1
Genes Upregulated by FGF-18

| Symbol | GenBank | Description | Ratio* |
|---|---|---|---|
| Impdh2 | NM_011830 | inosine 5′-phosphate dehydrogenase 2 | 2.32 |
| Sox11 | NM_009234 | SRY-box containing gene 11 | 3.82 |
| Rrm2 | NM_009104 | ribonucleotide reductase M2 | 3.67 |
| Calmbp1 | NM_009791 | calmodulin binding protein 1 | 3.42 |
| Pola1 | NM_008892 | polymerase (DNA directed), alpha 1 | 4.36 |
| BC006933 | NM_146040 | cDNA sequence BC006933 | 5.33 |
| Nola1 | NM_026578 | nucleolar protein family A, member 1 | 3.89 |
| | | (H/ACA small nucleolar RNPs) | |
| Sox4 | NM_009238 | SRY-box containing gene 4 | 2.21 |
| 2010317E03Ri | NM_026438 | RIKEN cDNA 2010317E03 gene | 2.95 |
| Nme4 | NM_019731 | expressed in non-metastatic cells 4, protein | 4.41 |
| Fignl1 | NM_021891 | fidgetin-like 1 | 2.6 |
| C330027C09Ri | NM_172616 | RIKEN cDNA C330027C09 gene | 3.32 |
| Etv1 | NM_007960 | ets variant gene 1 | 3.18 |
| Racgap1 | NM_012025 | Rac GTPase-activating protein 1 | 2.83 |
| Nol5a | NM_024193 | nucleolar protein 5A | 2.86 |
| Clu | NM_013492 | clusterin | 2.92 |
| Fgf18 | NM_008005 | fibroblast growth factor 18 | 31.42 |
| 6330403K07Ri | NM_134022 | RIKEN cDNA 6330403K07 gene | 8.25 |
| Basp1 | NM_006317 | brain abundant, membrane attached signal | 7.13 |
| | | protein 1 | |
| Anln | NM_028390 | anillin, actin binding protein | 4.18 |
| | | (scraps homolog, Drosophila) | |
| Prss11 | NM_019564 | protease, serine, 11 (Igf binding) | 5.83 |
| Meox1 | NM_010791 | mesenchyme homeobox 1 | 3.55 |
| Fabp7 | NM_021272 | fatty acid binding protein 7, brain | 4.49 |

*FGF-18/Control

TABLE 2
Genes Downregulated by FGF-18

| Symbol | GenBank | Description | Ratio* |
|---|---|---|---|
| Gyg1 | NM_013755 | glycogenin 1 | −2.68 |
| Tnni3 | NM_009406 | troponin I, cardiac | −3.73 |
| Actc1 | NM_009608 | actin, alpha, cardiac | −7.65 |
| Actc1 | NM_009608 | actin, alpha, cardiac | −3.07 |
| Mt2 | NM_008630 | metallothionein 2 | −4.24 |
| Lzp-s | NM_013590 | P lysozyme structural | −7.39 |
| Fmo1 | NM_010231 | flavin containing monooxygenase 1 | −8.54 |
| Crap | NM_013468 | cardiac responsive adriamycin protein | −8.24 |
| Mapt | NM_010838 | microtubule-associated protein tau | −4.47 |
| Rnase2 | NM_007895 | ribonuclease, RNase A family, 2 | −3.04 |
| C730036B01Ri | NM_178936 | RIKEN cDNA C730036B01 gene | −4.32 |
| Dpep1 | NM_007876 | dipeptidase 1 (renal) | −7.29 |
| 3732412D22Rik | BC042189 | RIKEN cDNA 3732412D22 gene | −5.08 |
| Igfbp6 | NM_008344 | insulin-like growth factor binding protein 6 | −6.61 |
| Vnn1 | NM_011704 | vanin 1 | −5.85 |
| A2m | NM_175628 | alpha-2-macroglobulin | −9.59 |
| Upk3b | NM_175309 | uroplakin 3B | −2.47 |
| 1190002H23Rik | NM_025427 | RIKEN cDNA 1190002H23 gene | −2.52 |
| Chia-pending | NM_023186 | chitinase, acidic | −4.59 |
| Pcdha4 | NM_007766 | protocadherin alpha 4 | −3.79 |
| Cyp4v3 | NM_133969 | cytochrome P450, family 4, subfamily v, polypeptide 3 | −4.44 |
| Pygm | NM_011224 | muscle glycogen phosphorylase | −2.44 |
| Fos | NM_010234 | FBJ osteosarcoma oncogene | −3.87 |
| Gadd45b | NM_008655 | growth arrest and DNA-damage-inducible 45 beta | −2.74 |
| Ager | NM_007425 | advanced glycosylation end product-specific receptor | −2.64 |
| Tna | NM_011606 | tetranectin (plasminogen binding protein) | −8.74 |
| Figf | NM_010216 | c-fos induced growth factor | −5.08 |
| Myb | NM_010848 | myeloblastosis oncogene | −10.46 |
| Eln | NM_007925 | elastin | −2.51 |
| Ly6a | NM_010738 | lymphocyte antigen 6 complex, locus A | −3.76 |
| Snca | NM_009221 | synuclein, alpha | −3.4 |
| Hdc | NM_008230 | histidine decarboxylase | −5.36 |
| C3 | NM_009778 | complement component 3 | −5.42 |
| 8430417G17Rik | NM_029415 | RIKEN cDNA 8430417G17 gene | −3.34 |
| Gsn | NM_010354 | gelsolin | −5.43 |
| Bcl2a1d | U23781 | B-cell leukemia/lymphoma 2 related protein A1d | −2.01 |
| Scd1 | NM_009127 | stearoyl-Coenzyme A desaturase 1 | −9.56 |
| Scgb1a1 | NM_011681 | secretoglobin, family 1A, member 1 (uteroglobin) | −12.69 |
| Angl | NM_007448 | angiogenin-like | −7.05 |
| Pr1 | NM_019432 | protein distantly related to the gamma subunit family | −2.94 |
| 1810015C04Rik | NM_025459 | RIKEN cDNA 1810015C04 gene | −5.25 |
| Pon1 | NM_011134 | paraoxonase 1 | −63.28 |
| Enpp2 | NM_015744 | ectonucleotide pyrophosphatase/phosphodiesterase 2 | −3.61 |
| 1600029D21Rik | NM_029639 | RIKEN cDNA 1600029D21 gene | −9.83 |
| Ctss | NM_021281 | cathepsin S | −2.48 |
| Slc34a2 | NM_011402 | solute carrier family 34 (sodium phosphate), member 2 | −4.82 |
| Klf4 | NM_010637 | Kruppel-like factor 4 (gut) | −2.36 |

*FGF-18/Control

### B. Results

1. Generation of SP-C-rtTA and (teto)₇CMV-FGF-18 Transgenic Mice

In the absence of doxycycline, double transgenic SP-C-rtTA and (teto)₇CMV-FGF-18 mice (heterozygous for each transgene) are viable. Fetal and postnatal single transgenic mice are produced in ratios predicted by Mendelian inheritance. Lung morphology is normal in both single transgenic mice and in the double transgenic mice in the absence of doxycycline. Prior in situ hybridization and reporter gene analyses of the lungs from SP-C-CAT and SP-C-rtTA mice, demonstrate that transgenic mRNA is selectively expressed in peripheral respiratory epithelial cells in the lungs of fetal and adult mice. Expression of firefly luciferase with the SP-C-rtTA system is observed as early as E10 in vivo. See Perl et al, Transgenic Res., (2002) 11:21-29. In adult SP-C-rtTA mice, rtTA mRNA is selectively expressed in peripheral conducting airways and type II epithelial cells. See Tichelaar et al, J. Biol. Chem., (2000) 275:11858-64 and Perl et al, Transgenic Res., (2002) 11:21-29. Two independent lines of (teto)₇CMV-FGF-18 target mice (lines A and B) are generated and mated to SP-C-rtTA mice. A similar morphologic phenotype is observed in the lungs of the independent (teto)₇CMV-FGF-18 lines after exposure to doxycycline. Subsequent studies utilized (teto)₇CMV-FGF-18 line “A.”

2. Conditional Expression of FGF-18 mRNA

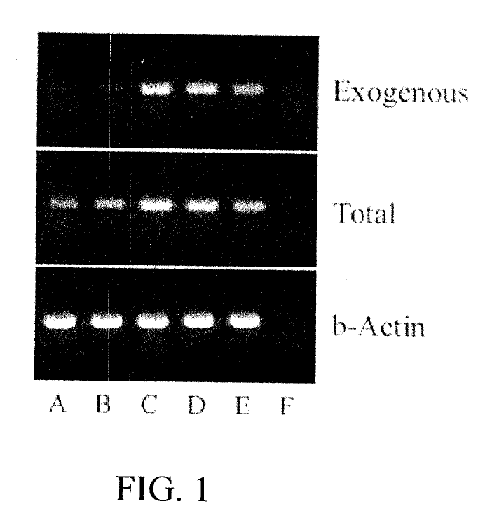

Transgene specific FGF-18 mRNA is assessed by RT-PCR in lungs of young adult mice, with and without addition of 0.5 mg/ml doxycycline in the drinking water. In adult double transgenic SP-C-rtTA×(teto)₇CMV-FGF-18 mice, FGF-18 mRNA is detectable at low levels in the absence of doxycycline, representing some “leak” in the absence of doxycycline, but is induced after oral doxycycline with the SP-C-rtTA mice. See Clark et al, Am. J. Physiol., (2001) 280:L705-15 and Tichelaar et al, J. Biol. Chem., (2000) 275:11858-64. Exposure of adult double transgenic mice to doxycycline did not alter lung morphology. In pups obtained from dams treated with doxycycline, FGF-18 mRNA is detected in fetal SP-C-rtTA×(teto)₇CMV-FGF-18 double transgenic mice, but is not readily detected in single transgenic animals. See lane A (wild type), lane B (single transgenic (teto)₇-FGF-18), lanes C-E (double transgenic), and lane F (double transgenic with no RT) of the Northern blot image in FIG. 1. Transgenic FGF-18 mRNA is not detected in other major organs of double transgenic mice, including liver, spleen, kidney, and brain, typical of the specificity of the SP-C promoter element, which is generally active only in respiratory epithelial cells in the lung. See Glasser et al, Am. J. Physiol., (1991) 261:L349-56. Exogenous FGF-18 mRNA is detected in testes of a double transgenic mouse on doxycycline, albeit at extremely low levels compared to that in lung.

5. Effects of FGF-18 on the Fetal Lung

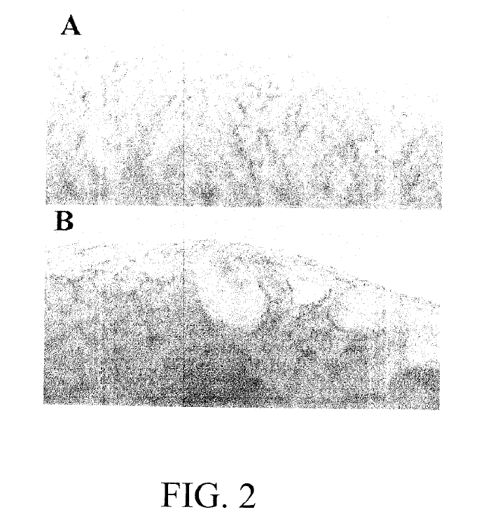

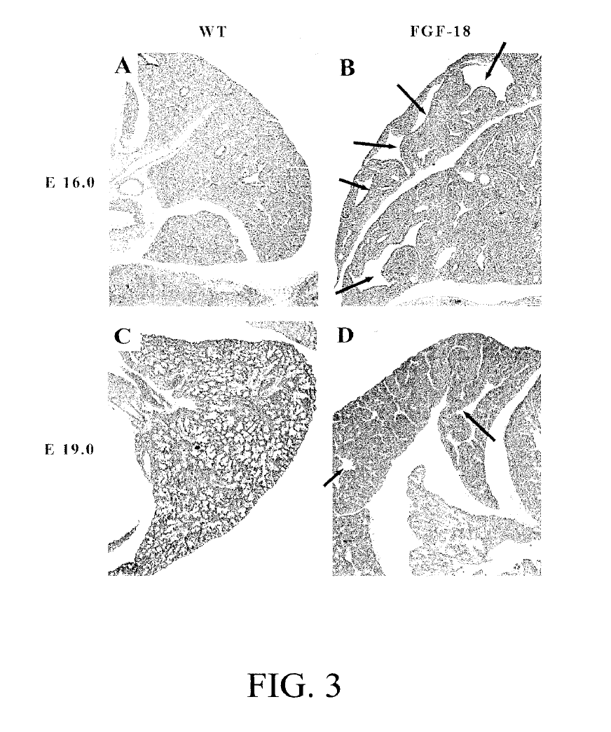

When dams are exposed on E6 and maintained on doxycycline during pregnancy, the percent survival of the offspring decreased to 50% of expected Mendelian numbers in double transgenic mice, consistent with lethality of the transgene. The structure of lungs from double transgenic SP-C-rtTA×(teto)₇CMV-FGF-18 offspring is assessed at E16-19. See FIG. 2 where marked abnormalities in the conducting airways and lung parenchyma are noted in FGF-18 expressing mice, while wild type embryos are unaffected. Arrows mark abnormal, large caliber, peripheral airways with features typical of the more proximal regions of normal lung. Without doxycycline, lung histology of double transgenic mice is generally indistinguishable from normal. In the presence of doxycycline, dramatic histologic abnormalities are observed in the lungs from double transgenic mice on E16-19. Branching morphogenesis is disrupted. A marked increase in length and caliber of conducting airways is observed. Decreased branching of peripheral airways with a marked reduction in peripheral saccules is consistently noted. The abnormal histology is not observed on E12.5, but is readily apparent at E16 and thereafter. Abnormally large airways and cysts are readily apparent in the FGF-18 expressing mice by direct visualization of the lung periphery. See FIG. 3 showing dissected lungs (E17) from double transgenic and control pups after exposure to doxycycline from E6 as photographed under a dissecting microscope, with arrows indicating dilated saccules.

Later in gestation, E18-19, stage specific sacculation of peripheral acinar buds and alveoli are lacking in the FGF-18 expressing mice. See FIG. 2. Elongated conducting airways of disordered caliber extended to the lung periphery. Atypical branching of bronchioles and distal bronchiolar-acinar tubules is observed, and normal acinar ducts and alveoli are markedly decreased or absent. The lung mesenchyme is thickened, containing few acinar tubules. Pulmonary blood vessels are prominent with abnormally large lumens, and alveolar capillaries are lacking. The residual peripheral airway saccules are not dilated and the normal alveolar structures of the newborn lung are lacking. Large, abnormal bronchial-like tubules, are observed throughout the lungs and many extended to the pleural surfaces. While similar abnormalities are observed in FGF-18 expressing fetal mice, variability in the extent of the histologic abnormalities is observed even in double transgenic mice from the same litter, suggesting that the timing, extent, or levels of transgene expression may influence the severity of the phenotype. Similar lung abnormalities are observed from litters capable of producing offspring with one copy or two copies of the CMV-FGF-18 transgene and in both A and B (teto)₇FGF-18 lines.

4. Aberrant Morphology and Differentiation of Epithelial Cells Lining the Lung Tubules

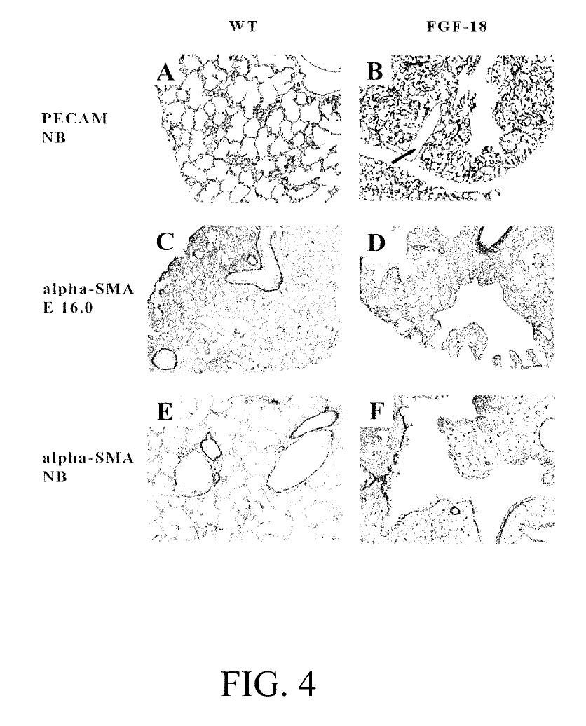

The peripheral conducting tubules in FGF-18 expressing mice at E16-19 are lined by a relatively homogenous population of columnar and cuboidal epithelial cells cilia. These abnormal airway epithelial cells stained intensely and homogeneously for TTF-1, reflecting a lack of terminal differentiation, and failure to form squamous cells (type I) in the periphery at E16-19 and newborns. See FIG. 4. ProSP-C and SP-B are detected at relatively low levels throughout the abnormal epithelium, consistent with the lack of both type II and squamous type I cell differentiation at E16 and 19. Atypical staining of surfactant proteins is observed in both basalar and apical regions of the cells while staining is detected in apical regions of type II cells in the normal lung. Abnormal clumps of cells staining for CCSP (Clara cell secretory protein) are observed in the elongated dilated respiratory bronchioles. However, CCSP is excluded from the most peripheral regions of lung tubules at E16 and 19 (see FIG. 4), as it is in the alveolar regions of the normal lung. The atypical cells lining lung tubules did not express Foxj1, a marker of ciliated cells in normal conducting airways, data not shown.

5. FGF-18 Altered Differentiation and Morphology of the Pulmonary Mesenchyme

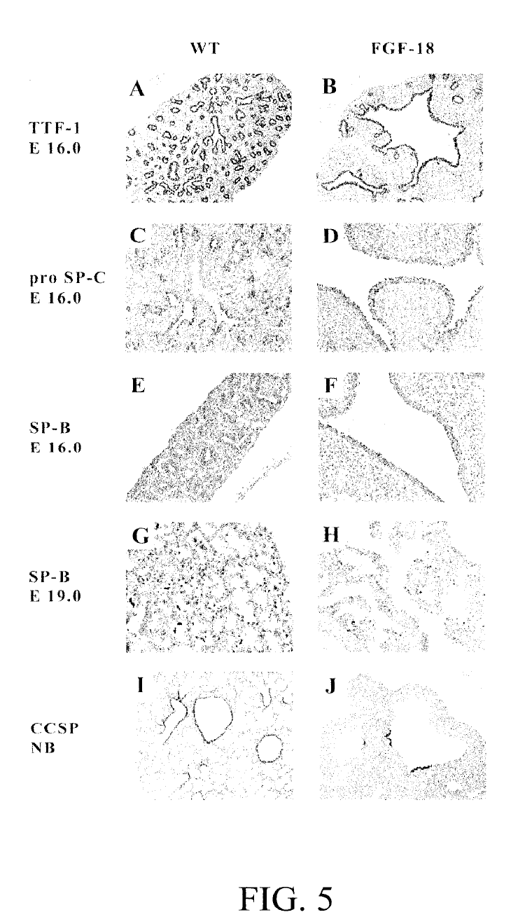

Pulmonary vascular development is perturbed as indicated by the abnormalities of PECAM staining in the pulmonary mesenchyme of the FGF-18 expressing mice. See FIG. 5. Extensive blood vessel development is noted in the abnormal mesenchyme surrounding the sparse, relatively small acinar tubules. Atypical pulmonary blood vessels in the periphery often had markedly enlarged lumenal diameter. See FIG. 5. α-Smooth muscle actin (α-SMA) staining, normally abundant in proximal, conducting airways and excluded from the alveolar region, is observed surrounding the aberrant airways in the lung periphery, being detected at sites that normally lack α-SMA staining in control littermates. See FIG. 5.

6. Ultrastructure of Fetal Lung from FGF-18 Expressing Mice

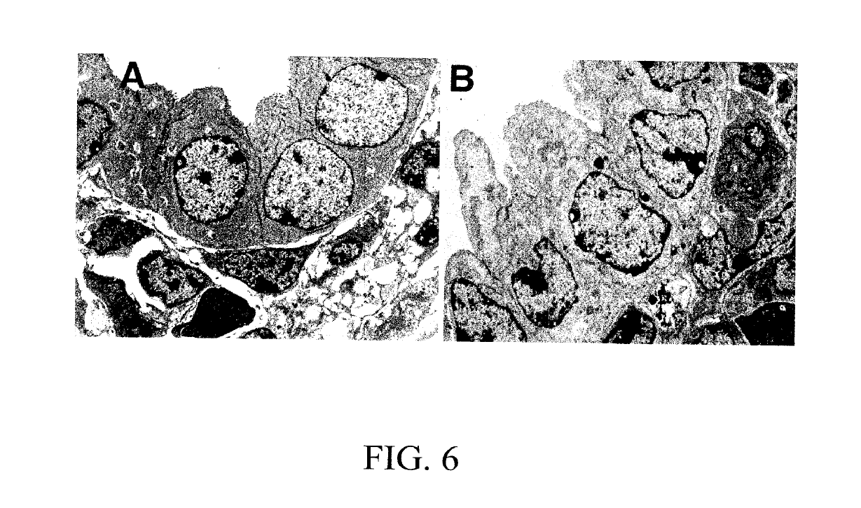

Ultrastructural analysis of lung tissue from FGF-18 expressing mice at E16 and 18 is consistent with observations at the light microscopic level. Abnormally large peripheral airways are lined by an atypical columnar epithelium. A relatively homogenous population of immature epithelial cells is observed in the peripheral tubules. Most of the terminal airspaces are lined by cuboidal or columnar cells and few squamous (type I) cells are observed. Epithelial cells are rich in glycogen, often lacked microvilli, and contained few lipid inclusions. See FIG. 6. Tubular myelin is not observed in the airways. Some of the atypical cells contained basal bodies, the latter typical of developing tracheal-bronchial ciliated cells. In lungs from control littermates, developing pre-type II cells are cuboidal and contained putative lamellar bodies. Tubular myelin is occasionally observed. At E18, squamous type I cells lined the most peripheral saccules in control pups. In FGF-18 expressing mice, the abnormal lung mesenchyme is poorly organized and contained abnormal blood vessels surrounded by prominent smooth muscle cells. Abnormal spaces are observed between the stromal cells in the pulmonary mesenchyme.

7. FGF-18 Perturbed Tracheal-Bronchial Cartilage

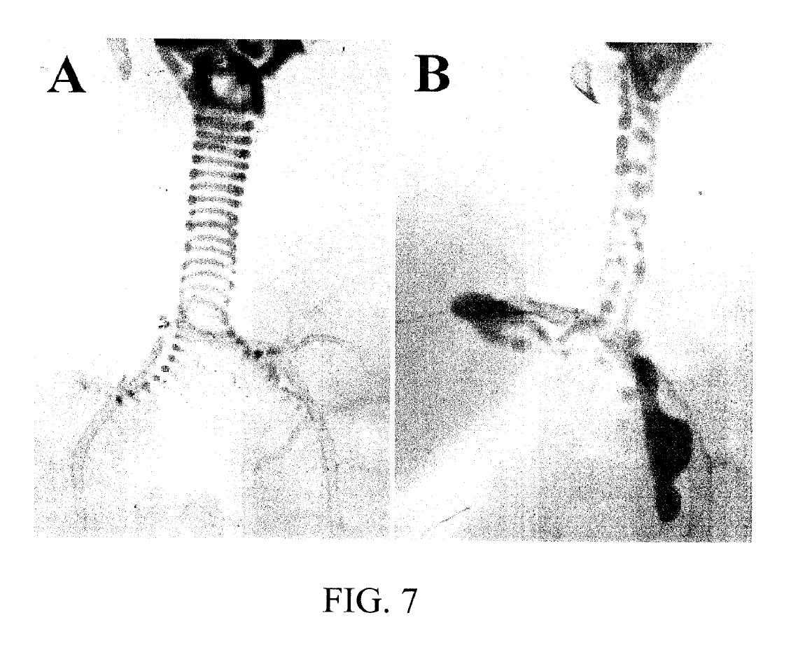

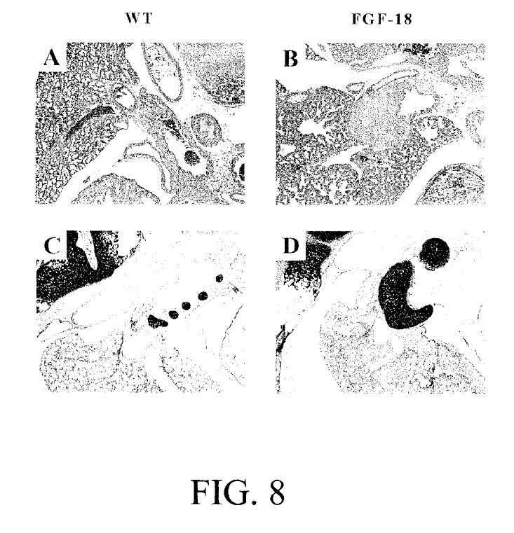

Consistent abnormalities in tracheal and bronchial cartilage are observed in the mice expressing FGF-18. Disordered size and shape of tracheal-bronchial cartilage rings and marked expansion of bronchial cartilage is consistently observed. See FIG. 7. Histologic analysis and procollagen II immunostaining demonstrates the abnormal bronchial cartilage that is readily detectable as early as E15-16.5, but not at E12.5. See FIG. 8

8. Effects of FGF-18 on FGF-10, BMP-4, and Sprouty-2 mRNAs

In whole mount in situ hybridization on lungs from E12 embryos, distribution and intensity of FGF-10, Sprouty-2, and BMP-4 mRNAs are not altered in FGF-18 expressing mice. Light Cycler® analysis for BMP-4, Sprouty-2, and FGF-10 mRNAs confirmed the lack of effect of FGF-18 on these mRNAs.

9. In situ Hybridization for Endogenous FGF-18 mRNA

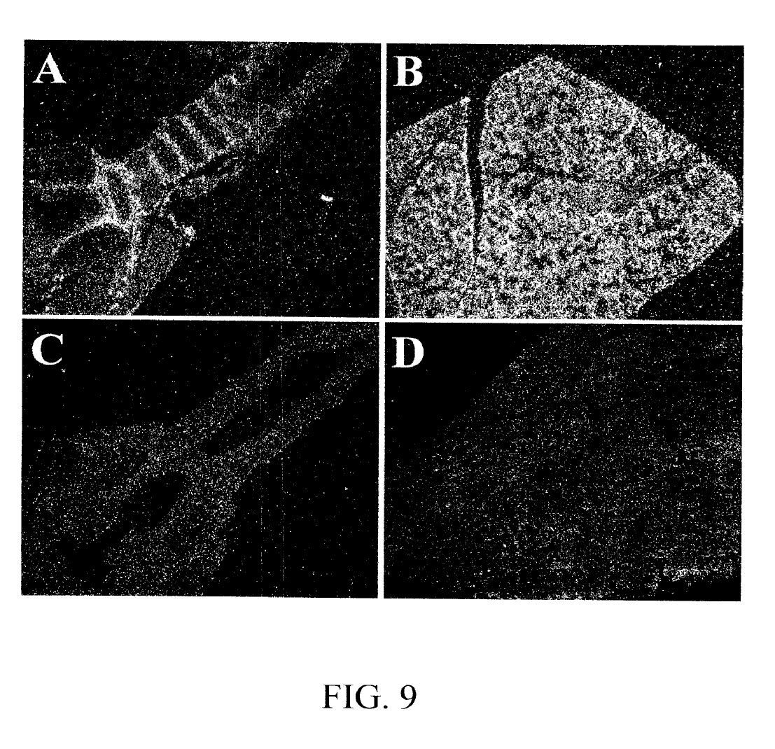

In situ hybridization with radiolabeled mouse FGF-18 antisense RNA demonstrated that FGF-18 mRNA is expressed at high concentrations in stromal cells surrounding tracheal-bronchial cartilage rings, in tissue surrounding laryngeal cartilage, and in the mesenchyme of the normal fetal lung from E12.5-18. See FIG. 9.

### C. Discussion

FGF-18 mRNA is conditionally expressed in respiratory epithelial cells of the lungs of fetal and postnatal mice. FGF-18 has little effect on the postnatal lung. However, lung morphogenesis is perturbed by expression of FGF-18 in the fetal lung. FGF-18 increases the length, caliber and disrupted branching of peripheral conducting airways, abnormal cytodifferentiation of epithelial cells lining the bronchial like lung tubules, blocks sacculation and alveolarization in late gestation, perturbs the organization of lung mesenchyme, increasing the extent and size of vascular structure and inhibiting capillary invasion of the lining epithelium, and induces cartilage in the periphery of main bronchi. Taken together, FGF-18 influences various aspects of proximal-distal programming of the lung, enhancing elements of the conducting airways and inhibiting those of the lung periphery.

1. Effects of FGF-18 on Lung Structure

FGF-18 produces a homogenous cuboidal-columnar epithelium that lacked features characteristic of normal, peripheral tubules. The atypical columnar epithelial cells are rich in glycogen, and lacked other features typical of type II cells. Squamous cell differentiation is inhibited. On the other hand, some aspects of proximal epithelial cell differentiation are not apparent in the abnormal epithelial cells. Neither cilia, Foxj1 nor CCSP staining are observed in most of the atypical epithelial cells in the peripheral lesions induced by FGF-18. Likewise, FGF-18 does not alter the levels or sites of expression of FGF-10, BMP-4, and Sprouty-2 mRNAs, suggesting that the effects of FGF-18 on lung morphology are not mediated via these pathways.

The sites and levels of ectopic expression of FGF-18 may influence the observed morphological effects of the FGF-18 transgene. Since FGF-18 is expressed in epithelial cells and not in mesenchymal cells, as in wild type mice, bioavailability of the ligand or accessibility of the ligands to FGF receptors, may be distinct in the transgenic mice. In situ hybridization for the endogenous FGF-18 mRNA confirms its expression in the pulmonary mesenchyme and demonstrated its distribution surrounding forming cartilage rings in the trachea and bronchi. This site of expression is consistent with a potential role for FGF-18 in cartilage formation.

2. Increased Cartilage Formation

Expression of FGF-18 perturbs cartilage ring morphology in the trachea, and expands cartilaginous tissue in peripheral regions of main bronchi. The presence of endogenous FGF-18 mRNA surrounding normal cartilage rings in the developing trachea is also consistent with the role for FGF-18 and FGF-R signaling in tracheal-bronchial cartilage morphogenesis. Ectopic cartilage is not seen on E12.5, but is readily apparent at E16. Abnormalities in cartilage are not observed when FGF-18 is expressed postnatally. The shape and contiguity of cartilaginous rings are perturbed at normal sites of tracheal-bronchial cartilage formation; however, large amounts of cartilage formed in the distal bronchi of the FGF-18 expressing mice. These findings support the concept that FGF-18 altered proliferation of chondrocytes or prechondrocytes at critical times during morphogenesis. The abnormal cartilaginous tissue stained intensely for collagen type II, an early marker of cartilage differentiation.

Abnormalities in differentiation of the epithelial cells lining the conducting airways of the FGF-18 transgenic mice included atypical columnar morphology, the lack of saccular-alveolar differentiation, and is associated with a striking inhibition of type I and type II epithelial cell differentiation. Squamous type I cells and capillary invasion of peripheral tubules are lacking. Homogenous staining for TTF-1, proSP-C, and SP-B in peripheral epithelial cells and inhibition of differentiation of type I cells seen at the ultrastructural level are consistent with a failure of terminal differentiation of the peripheral lung parenchyma. However, characteristics of the abnormal epithelial cells are not consistent with a transformation of peripheral cells to a proximal epithelial cell type. While some of these abnormal cells contained basal bodies, usually associated with ciliated cells in proximal regions of the lung, the atypical cells in the most peripheral lesions did not express CCSP or contain numerous cilia, markers of normal mouse conducting airways. See Wert et al, Dev. Biol., (1993) 156:426-43 and Tichelaar et al, J. Histochem. Cytochem., (1999) 47:823-31. SP-B and proSP-C are localized within the atypical columnar cells in both basal and apical regions of the cells, a pattern not seen in normal alveolar type II cells, wherein these proteins are normally concentrated in apical membranes. Thus, changes in epithelial differentiation do not represent a complete proximalization of the peripheral respiratory epithelium, the abnormal cells sharing features of immature proximal and peripheral respiratory epithelial cells.

### D. Conclusions

This experiment and resultant findings demonstrate that increased expression of FGF-18 markedly increases the length and caliber of conducting airways, and altered branching of the bronchial tree in peripheral regions of the fetal lung. Formation of acinar and alveolar elements that are normally lined by cuboidal and squamous epithelial cells is inhibited by FGF-18. Increased caliber of peripheral pulmonary blood vessels and lack of alveolar capillaries are consistent with enhancement of features of proximal lung structures and inhibition of alveolarization. FGF-18 induces ectopic cartilage and altered blood vessel formation and induced α-smooth muscle actin, consistent with the enhancement of development of proximal rather than peripheral regions of the lung. Effects of FGF-18 are limited to the fetal lung, and are not observed when the transgene is activated postnatally, supporting the concept that FGF-18 influences cell proliferation, differentiation or migration earlier in morphogenesis. FGF-18 has unique effects on lung formation, preferentially shifting some developmental and morphogenetic programs of blood vessels, cartilage, and airways towards proximal programs.

The following example illustrates the involvement of Sonic Hedgehog (Shh), a target gene regulated by FGF-18 expression, in the formation of cartilage:

### A. Experimental Procedures

1. Animal Use and Doxycycline Administration

Animals are housed in a pathogen-free vivarium in accordance with institutional policies. Detection of a vaginal plug and correlating pup weights determines gestational age. Doxycycline is administered to dams in food (625 mg/kg; Harlan Teklad, Madison, Wis.) at various times during gestation, and crosses the placental barrier to treat fetal pups.

2. Generation of Transgenic Mice

Abbreviations for mice used in this example are shown in Table 3 below. Breeding strategies for mice are shown in Table 4 below. Transmission of all transgenes exhibits typical Mendelian inheritance patterns:

TABLE 3
Description of Transgenic Mice

| Transgenic Mice | Label | Description |
|---|---|---|
| Shh^{flx/flx} | a | Shh gene has loxP sites flanking exon |
| Shh−/− | b | Shh null mutant mouse |
| SP-C-rtTA^{tg} | c | Transactivator mice; directs transgene |
| | | expression to respiratory epithelium |
| (tetO)₇CMV-Cre | d | Expression of Cre recombinase enzyme |
| | | with doxycycline treatment |
| (tetO) ₇CMV- | e | Expression of rat Shh with doxycycline |
| rShh | | treatment |
| Shh^{+/+} or Shh^{+/-} | | Wild-type control littermates generated |
| | | from Shh null mutants |

TABLE 4
Breeding Strategies

| Transgenic Breeding | | |
|---|---|---|
| Strategy | Product | Abbreviation |
| a × b | shh^{flx/-} | none |
| a × c | SP-C-rtTA^{tg}; Shh^{flx/flx} | none |
| (a × b) × d | (tetO)₇CMV | none |
| ((a × b) × d) × (a × c) | SP-C-rtTA^{tg}; (tetO)₇CMV- | Shh^{flx/-}(Shh^{Δ/-}**) |
| | Cre^{tg/tg}; Shh^{flx/-} | Shh^{flx/flx} |
| | or | (Shh^{Δ/Δ}**) |
| | SP-C-rtTA^{tg}; (tetO)₇CMV- | |
| | Cre^{tg/tg}; Shh^{flx/flx} | |
| b × e | Shh^{+/-}; (tetO)₇CMV-rShh^{tg} | none |
| c × e | SP-C-rtTA^{tg}; (tetO)₇CMV-rShh^{tg} | none |
| (b × e) × (c × e) | SP-C-rtTA^{tg}; Shh^{+/-}; (tetO)₇CMV- | none |
| | rShh^{tg/tg} | |
| ((b × e) × (c × e)) × (b × e) | SP-C-rtTA^{tg}; Shh^{-/-}; (tetO)₇CMV- | Shh^{rescue} |
| | rShh^{tg/tg} | |

**Shh^{flx/flx} and Shh^{flx/-} mice become Shh^{Δ/Δ} and Shh^{Δ/-} respectively, when treated with doxycyline where Δ indicates that recombination has occurred.

a. Shh^{flx/fl}, or Shh^{flx/-} and Shh^{Δ/Δ} or Shh^{Δ/-} Mice

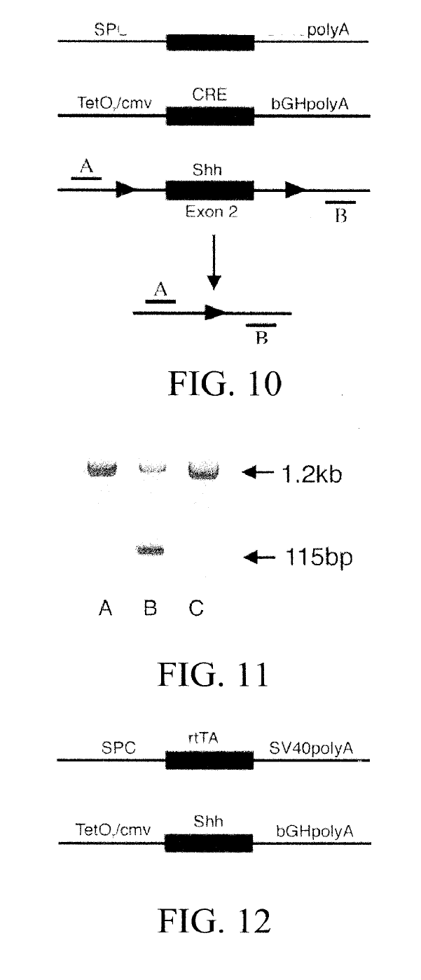

FIG. 10 shows linear maps of a transactivator construct that directs expression of a reverse tetracycline transactivator protein (rtTA) to the peripheral epithelial cells of the lung using the human 3.7 kb SP-C promoter (upper map above vertical arrow); an expression construct where the gene for Cre-recombinase (CRE) is linked to the (tetO)₇CMV promoter (intermediate map above vertical arrow); and a targeting construct where two identically oriented loxP sites have been inserted on either side of exon 2 of the Shh gene (lower map above vertical arrow). These constructs are used to generate founder mice which, when mated to produce compound mutant mice, undergo lung-specific recombination at the Shh exon 2 locus following administration of doxycycline (product, below arrow).

Transgenic mice bearing loxP sites flanking exon2 of the Shh gene (Shh^{flx/flx}) are provided by A.P. McMahon (see Pepicelli et al, Curr. Biol., (1998) 8:1083-1086). Shh^{flx/flx} are bred to Shh^{+/-} mice and then mated to SP-C-rtTA mice that express the rtTA gene under control of the human SP-C promoter (see Tichelaar et al, J. Biol. Chem., (2000) 275:11858-64). Transgenic (tetO)₇CMV-Cre recombinase mice generated by pronuclear injection (see Perl et al, Transgenic Res., (2002) 11:21-29) are crossed to SP-C-rtTA, Shh^{flx/flx}, or Shh^{flx/-} mice to produce double transgenic mice. Triple transgenic mice result from crosses of SP-C-rtTA^{tg}; Shh^{flx/flx} and (tetO)₇CMV-Cre^{tg/tg}; Shh^{flx/-}. Transgenic animals possessing the SP-C-rtTA gene, the (tetO)₇CMV-Cre recombinase gene and homozygous floxed alleles (Shh^{flx/flx}) or one floxed allele and one null allele (Shh^{flx/-}) become Shh^{Δ/Δ} or Shh^{Δ/-} upon treatment with doxycycline.

Primers used for detection of normal, floxed, and null alleles are represented by A and B in FIG. 10. PCR analysis is performed, using primers A and B, on mRNA extracted from heart (lane A) and lungs (lane B) removed from Shh^{Δ/-} mice treated with doxycycline throughout gestation, as shown in FIG. 11. mRNA extracted from lungs of Shh flx/− mice that is not treated with doxycycline (lane C) is also analyzed for comparison.

b. SP-C-rtTA^{tg}; (tetO)₇CMV-rShh^{tg} Mice

The rat Shh cDNA is inserted between the (tetO)₇CMV promoter and the 3′-untranslated region of the bovine growth hormone (bGHpolyA) gene as described by Tichelaar et al, J. Biol. Chem., (2000) 275:11858-64 and Clark et al, Am. J. Physiol., (2001) 280:L705-15. The construct is injected into oocytes from FVB/N mice and Southern blotting is used to identify founders. Offspring are screened by Southern blot and PCR analysis. Mice transmitting the (tetO)₇CMV-rShh^{tg} transgenes are bred to SP-C-rtTA FVB/N transgenic mice as described by Tichelaar et al, J. Biol. Chem., (2000) 275:11858-64 and Clark et al, Am. J. Physiol., (2001) 280:L705-15 to produce SP-C-rtTA^{tg}; (tetO)₇CMV-rShh^{tg} compound mutant mice.

c. Shh^{rescue} Mice

FIG. 12 shows a linear map of rat Shh cDNA inserted between the (tetO)₇CMV promoter and bGHpolyA (upper). This construct is used to constitutively express SHH in the lungs of the Shh^{-/-} mouse when mated to mice bearing the SP-C-rtTA; (tetO)₇CMV-rShh construct (lower). Treatment with doxycycline results in expression of Shh in peripheral respiratory epithelial cells of the Shh^{-/-} mice.

Shh null mutant (Shh^{-/-}) mice are provided by A.P. McMahon (see Pepicelli et al, Curr. Biol., (1998) 8:1083-1086). Shh^{+/+} and Shh^{+/-} mice exhibit no observable abnormalities. Expression of the rat Shh cDNA in Shh^{-/-} mice results from mating Shh^{+/-} mice with SP-C-rtTA^{tg}; (tetO)₇CMV-rShh^{tg/tg} compound mutant mice to produce transgenic mice in a Shh^{+/-} background, which are then crossed to Shh^{+/-}; (tetO)₇CMV-rShh^{tg/tg} compound mutant mice to produce the double transgenic “rescue” mice in a Shh^{-/-} background, herein termed Shh^{rescu}.

d. Patched-1/^{LacZ/+} Mice

Heterozygous Ptch1^{LacZ/+} mice in which β-galactosidase is expressed under control of the Ptch1 promoter are obtained from Jackson Labs (see Goodrich et al, Dev. Biol., (1997) 211:323-34 and Science, (1999) 277:1109-13). β-Galactosidase activity is developed in lungs and trachea taken from Ptch1^{LacZ/+} mice at E11.5, 13.5, and 15.5. The lungs and tracheas are then embedded in paraffin and sectioned for microscopy.

3. Histology, Immunohistochemistry, In situ Hybridization, and Electron Microscopy

Fetuses are removed by hysterectomy after the dams are killed by lethal injection with 0.25 ml of anesthetic (ketamine, xylazine, acepromazine). The fetuses are weighed, their chests opened, and the lungs are removed. The lungs are placed in 4% paraformaldehyde at 4° C., and weighed the following day. After the lungs are visually assessed for gross abnormalities, the tracheas are removed and placed in 95% ethanol for whole mount alcian blue staining of cartilage. The remaining lung is dehydrated in an ascending series of alcohols, infiltrated and embedded in paraffin. Lungs with attached tracheas and hearts are also embedded. Tissue sections are immunostained for surfactant protein-C propeptide (proSP-C; Chemicon), Clara cell secretory protein (CCSP), FOXJ1, β-tubulin IV (Biogenix, mouse monoclonal, clone# ONS1A6), platelet endothelial cell adhesion molecule (PECAM; Pharmagen, rat monoclonal, clone #CD31), α-smooth muscle actin (α-SMA; Sigma, mouse monoclonal, clone# NA4), calcitonin gene-related protein (CGRP; Sigma-Aldrich), T1-a (University of Iowa Hybridoma Bank, mouse monoclonal, clone# 8.1.1), SHH-N (Santa Cruz) and thyroid transcription factor-1 (TTF-1) as described by Tichelaar et al, J. Biol. Chem., (2000) 275:11858-64; Clark et al, Am. J. Physiol., (2001) 280:L705-15; Watkins et al, Nature, (2003) 422:313-17; and Unger et al, Am J Path, (2003) 162:547-55. Proliferation is assessed by immunostaining sections from control and transgenic lungs, at E13.5 and E18.5 for α-phosphohistone H3 (αPH3; US Biological), a mitotic cell marker. For electron microscopy, tissue is fixed, processed and evaluated as described by Clark et al, Am. J. Physiol., (2001) 280:L705-15. In situ hybridization for VegfA and Ptch1 mRNA is performed using ³⁵S-UTP-labeled riboprobes as described by Wert et al, Dev. Biol., (1993) 156:426-43 and Greenberg et al, Dev. Dyn., (2002) 224:144-53. Whole mount in situ hybridization for Fgf10 mRNA is performed at E12.5-13.5 using digoxigenin-labeled riboprobes. See Wilkinson, In Situ Hybridization: A Practical Approach, (1998) (New York: Oxford University Press).

4. RNA Isolation, Preparation and Analysis Using Affymetrix Analysis

Total lung RNA from Shh^{Δ/Δ} and Shh^{Δ/-} mice and control littermates (Shh^{flx/flx}, Shh^{flx/-}) at E13.5 is collected for microarray hybridization using Mouse Expression Set 430AgeneChip (Affymetrix, Inc). Eight chips from four pair-wise designed experiments are used. Affymetrix MicroArray Suite version 5.0, JAM4 (SAS Institute, Inc.) and GeneSpring 5.0 (Silicon Genetics, Inc.) are used for data analysis. Hybridization data are sequentially subjected to normalization, transformation, filtering and functional classification as described by deFelice et al, J. Biol. Chem., (2003) 278:35574-83. Genes differentially expressed between Shh^{Δ/-} and control mice (Shh^{flx/-}) are identified by Student t-test at P value <0.05 and a fold change of 1.5. To evaluate data consistency and reproducibility, coefficient of variation among replicates is calculated with the maximal cutoff line at 50%. In addition, a count and percentage analysis, using the Affymetrix Change Call algorithm for all possible combinations of comparison between Shh^{Δ/-} and the control (16 combinations), is used as an additional filter with the minimal cut off line as 14 of 16 combinations.

5. Statistical Analysis

Sections of lung from Shh^{Δ/-} and control (Shh^{flx/-}) mice are immunostained for αPH3 and the number of mitotic nuclei is assessed for both epithelial (cells/mm length) and mesenchymal (cells/mm²) cell compartments using Metamorph Imaging software (Universal Imaging Corporation). Measurements for the epithelium exhibit a non-parametric distribution and therefore the Mann-Whitney Rank Sum test is performed. T-test analysis is carried out for the mesenchymal measurements since the data exhibit a normal distribution. All statistics for this analysis are carried out using SigmaStat software (SPSS Inc.).

For each pup, lung-to-body weight ratios are calculated and tested for significance using 2-Way ANOVA with pup-within-each-litter treated as a blocking factor. The gross appearance of lungs removed from the pups is visually assessed for various characteristics, such as lobe formation, lung hypoplasia, presence of cysts, peripheral tubule dilation, and tracheal abnormalities, and assigned a composite score. Statistical significance is determined using the Kruskall-Wallis procedure (SAS Software, NPAR1WAY; SAS Institute, Inc.).

### B. Results

1. Comparison of Transgenic Pup Phenotypes, Lung, Morphology, and Histology after Treatment with Doxycycline

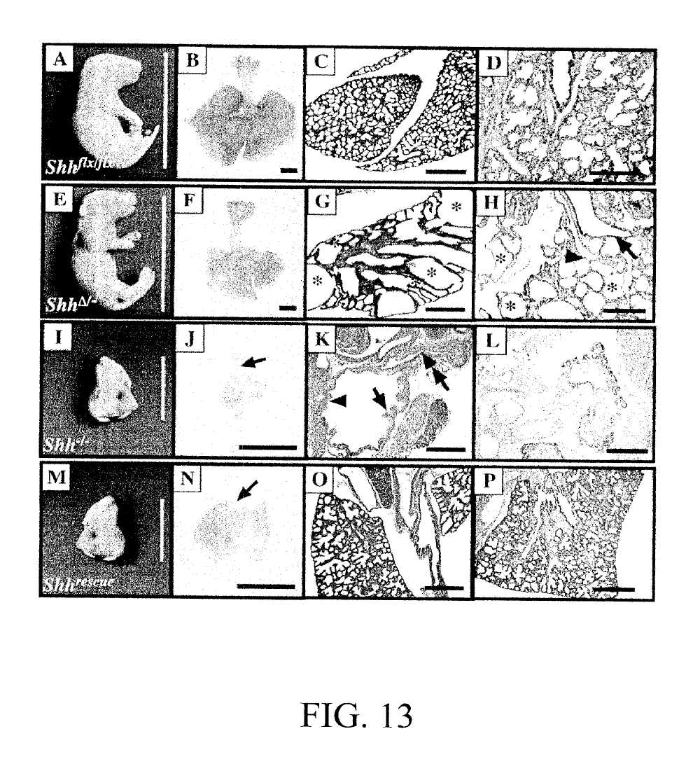

As shown in FIG. 13 (n>5 for each genotype; scale bar=1.2 cm for A, E, I and M, 1 mm for B, F, G, J and N, 200 mm for C, G, H, K, L, O, and P, 100 mm for D), littermate control Shh^{flx/flx} mice (see A) and Shh [symbol] ^{/-} (see E) pups are indistinguishable at E18.5, while Shh^{-/-} (see I) and Shh^{rescue} (see M) pups are grossly abnormal. Lungs from Shh^{Δ/-} (see F) pups are hypoplastic with fluid-filled cysts and dilated peripheral tubules. Lungs from Shh^{-/-} (see J) and Shh^{rescue} (see N) pups are severely hypoplastic compared to control littermates (see B), but significant restoration of peripheral lung tissue is observed in lungs from Shh^{rescue} (N) pups. Sections of lung are stained with hematoxylin and eosin (C, G, K and O) or are immunostained for SHH-N (D, H, L and P). Lungs from Shh^{flx/flx} mice (C) are histologically normal with SHH-N immunostaining detected in both respiratory and conducting epithelium (see D). The dilated peripheral tubules of the lung from Shh^{Δ/-} mice (see “*” in G) correlate with areas of reduced or absent SHH-N immunostaining (see arrowhead in H), while immunostaining for SHH-N is still detected in the conducting airway epithelium (see arrow in H). SHH-N is detected in the peripheral areas of the lung from Shh^{-/-} mice (L), indicating detection of a truncated mutant SHH peptide. In Shh^{rescue} mice (O), branching morphogenesis is restored in association with immunostaining for SHH-N (P). Competitive binding experiments with SHH blocking peptide demonstrate that the immunostaining for SHH in Shh^{-/-} mice is ablated by preabsorption with the SHH peptide.

2. Pulmonary Morphology, Histology and Proliferation

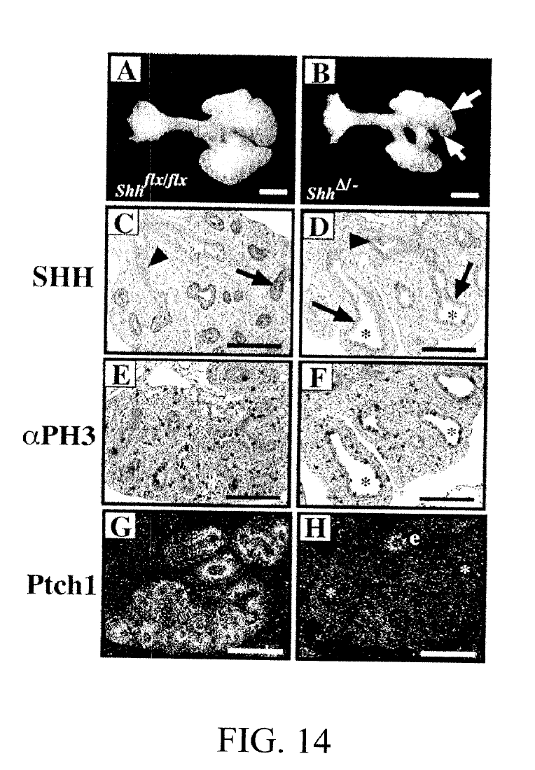

Dams are treated with doxycycline from E0.5-13.5 and lungs are harvested at E13.5. As shown in FIG. 14 (n>5 for each genotype; scale bar=1 μm for A and B, 100 μm for C—H), when compared with Shh^{flx/flx} control littermates (A), lungs from Shh^{Δ/-} mice (B) are hypoplastic with reduced branching morphogenesis, as indicated by dilated peripheral tubules (see “*” in D). SHH-N immunostaining is not detected in proximal tubule epithelium (see arrowheads in D) and is reduced or absent from the epithelium of the abnormally dilated peripheral tubules compared to control (see arrows in C). While the size of the lung is decreased in Shh^{Δ/-} mice, a-phosphohistone H3 (αPH3) immunostaining is not altered (see E and F). In situ hybridization indicates reduced levels of Ptch1 mRNA in Shh^{Δ/-} mice (see H) compared to control (see G).

3. Immunostaining for Epithelial Cell Markers

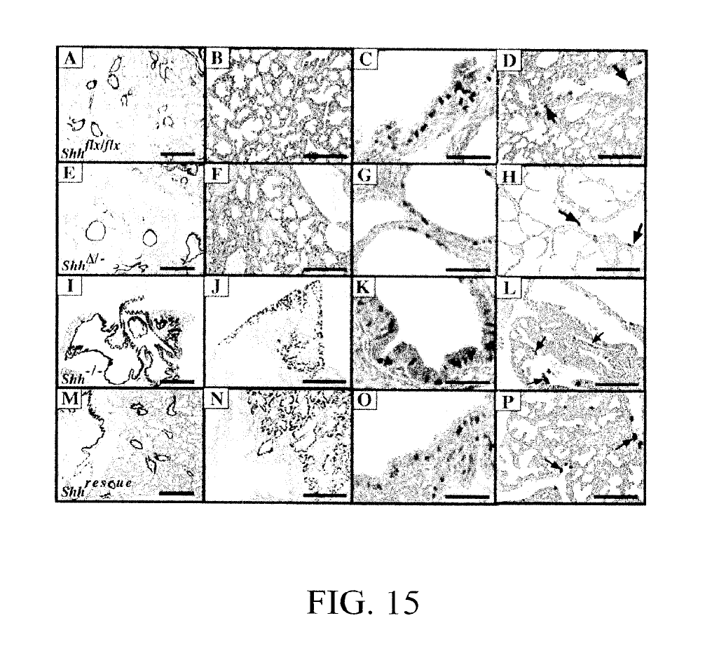

As shown in FIG. 15 (n=3 or greater per genotype for each marker; scale bar=200 μm for A, E-F, I-K and M-N, 100 μm for B, 50 μm for C, G and O, and 100 μm for D, H, L and P), lungs from Shh^{flx/flx} (see A-D), Shh^{Δ/-} (see E-H), Shh^{-/-} (se I-L) and Shh^{rescue} (see M-P) mice at E18.5 are immunostained for CCSP, proSPC, FOXJ1, and CGRP. Epithelial differentiation selective markers are detected in lungs from all mice examined.

4. Shh Influences on Pulmonary Vascular Morphogenesis

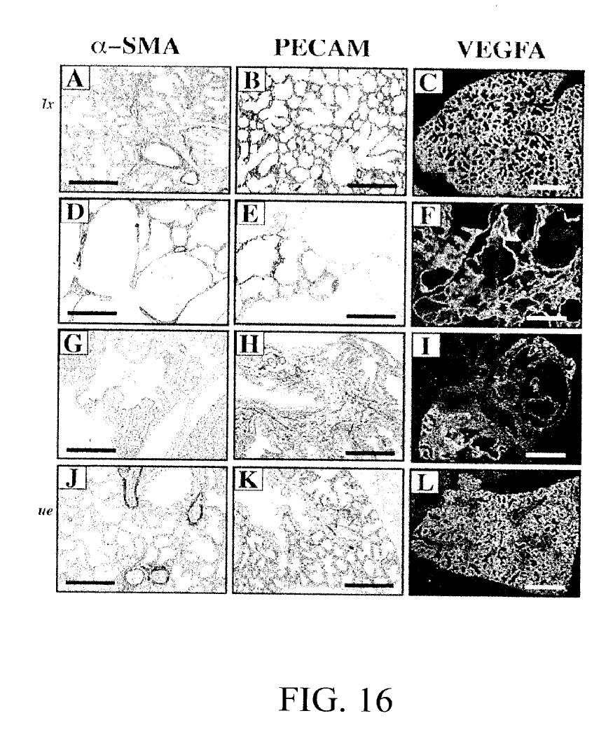

As shown in FIG. 16 (n=3 or greater per genotype for each marker; scale bar=100 [symbol] m for A-B, D-E, G-H and J-K, and 200 [symbol] m for C, F, I and L), lungs from Shh^{flx/flx} (see A-C), Shh^{Δ/-} (see D-F), Shh^{-/-} (see G-I), and Shh^{rescue} (see J-L), mice at E18.5 are immunostained for [symbol] -SMA or PECAM, or hybridized with radiolabeled riboprobe for VegfA mRNA. [symbol] -SMA staining is decreased in Shh^{Δ/-} lungs (see D), but is absent in Shh^{-/-} lungs (see G), indicating that differentiation or survival of pulmonary smooth muscle precursors is dependent upon SHH. PECAM staining is markedly reduced in the distal lung from the Shh^{Δ/-} (see E) and Shh^{-/-} mice (see H). VegfA mRNA is decreased in the peripheral respiratory epithelium of Shh^{-/-} mice (see I) but is variably detected in Shh^{Δ/-} mice (see F).

5. RNA Analysis of Lung Tissue from Shh^{Δ/Δ} and Shh^{Δ/-} Mice

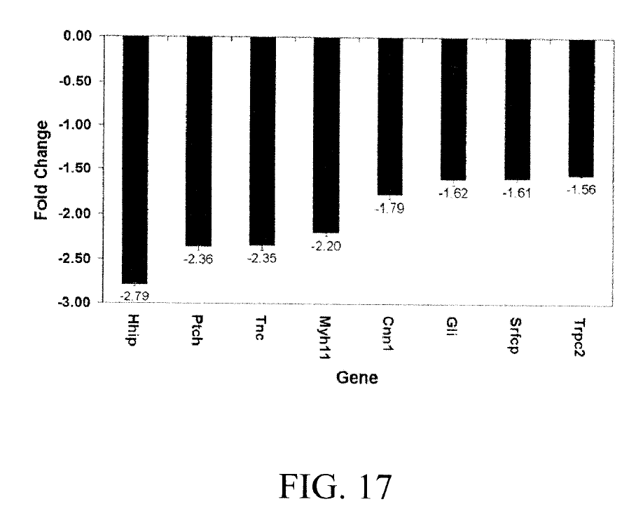

Microarray hybridization and subsequent data analysis are performed using total lung RNA from Shh^{Δ/Δ} and Shh^{Δ/-} mice and their Shh^{flx/flx} and Shh^{flx/-} control littermates at E13.5. Twenty RNAs are decreased by 50% (1.5 fold) (see FIG. 17 and Table 5). In Shh^{Δ/-} mice, RNA encoded by genes in the SHH pathway, including Ptch1, Gli1 and Hip1, is decreased significantly (see FIG. 17 and Table 5 below). These genes are components of the SHH signaling pathway and are known to be regulated by SHH. RNAs from a number of genes that are selectively expressed in smooth muscle cells (Tnc, Myh11, Cnn1, Stfcp and Trpc2) are also decreased (see FIG. 17 and Table 5 below), consistent with decreased a-SMA immunostaining and supporting the reduction in vascular and bronchial smooth muscle observed in these mice. Additional genes that exhibited increased expression patterns when lungs from Shh^{Δ/Δ} and Shh^{Δ/-} mice are compared with control animals are listed in Table 4 below. The bar graph in FIG. 17 represents the average fold decrease (±SEM) in selective lung mRNA levels in Shh^{Δ/Δ} and Shh^{Δ/-} mice versus their Shh^{flx/fl} and Shh^{flx/-} control littermates. Mean and standard error are calculated from four independent hybridizations:

TABLE 5
Genes Downregulated in Lungs from Shh^{Δ/-} Mice at E13.5

| Symbol | GenBank | Description | Ratio |
|---|---|---|---|
| Hhip | BB773386 | hedgehog-interacting protein | −2.09 |
| Cnn1 | NM_009922 | calponin | −1.76 |
| Tagln | BB114067 | transgelin | −1.70 |
| Ednrb | BB770914 | endothelin receptor type B | −1.77 |
| A230106A1Rik | BG071079 | RIKEN cRNA A230106A15 gene | −1.89 |
| Mgll | AK006949 | monoglyceride lipase | −1.68 |
| Sftpc | NM_011359 | surfactant associated protein C | −2.17 |
| Myh11 | BC026142 | Myosin heavy chain 11 | −2.18 |
| | BI143915 | Mus musculus, clone IMAGE: 3670705 | −1.93 |
| Srfcp-pending | AF38055 | SRF co-factor protein | −1.61 |
| Gli1 | NM_010296 | GLI-Kruppel family member GLI1 | −1.60 |
| Mgll | BI411560 | monoglyceride lipase | −1.65 |
| Smoc2 | NM_022315 | SPARC related modular calcium binding | −1.55 |
| Acta2 | NM_007392 | actin, alpha2 | −1.55 |
| Enc1 | BM120053 | ectodermal-neural cortex 1 | −1.55 |
| Trpc2 | AK007972 | myosin, light polypeptide 9 | −1.56 |
| Actg2 | NM_009610 | actin, gamma 2 | −1.59 |
| 4933425F03Rik | BC16096 | RIKEN cDNA 4933425F03 gene | −1.60 |
| Adamdec1 | NM_021475 | ADAM-like, decysin 1 | −3.82 |
| Hhip | NM_020259 | hedgehog-interacting protein | −2.78 |
| Ptch | NM_008957 | patched homolog tenascin C | −2.33 |
| Tnc | NM_011607 | | −2.29 |
| Dio2 | NM 010050 | deiodinase, iodothyronine, type II | −2.08 |

TABLE 6
Genes Unregulated in Lungs from Shh^{Δ/-} Mice at E13.5

| Symbol | GenBank | Description | Ratio |
|---|---|---|---|
| Dex | BM115908 | doublecortin | 1.65 |
| MGC38363 | BC025461 | superfamily transmembrane 4 member 3 | 1.68 |
| Ubce8 | BB042892 | RIKEN cDNA 6030424L22 | 1.73 |
| Epha3 | M68513 | Eph receptor A3 | 1.77 |
| Prrx1 | BQ175427 | paired related homeobox1 | 1.82 |
| Tnfsf13b | NM_03362 | tumor necrosis factor (ligand) superfamily, member 13b | 1.84 |
| C76683 | BE949497 | EST's, moderately similar to putative c-Myc-responsive | 1.85 |
| Klf5 | BG069607 | Kruppel-like factor 5 | 1.89 |
| Egfl6 | NM_01939 | EGF-like-domain, multiple 6 | 1.94 |
| Afp | NM_00742 | alpha fetoprotein | 1.96 |
| Slc15a2 | BC018335 | solute carrier family 15 (H⁺/peptide transporter) member 2 | 1.97 |
| LOC219134 | BC024118 | hypothetical protein BC024118 | 1.99 |
| LOC216019 | BC016235 | similar to hexokinase1, isoform HKI-R | 2.00 |
| Dkk2 | NM_02026 | dickkopf homolog 2 (Xenopus laevis) | 2.02 |
| 2410012A13Rik | NM_02339 | RIKEN cDNA 2410012A13 | 2.03 |
| Pla2g1b | AV060866 | phospholipase A2, group 1B, pancreas | 2.04 |
| Anp32a | AF022957 | acidic (leucine-rich) nuclear phosphoprotein 32 family | 2.06 |
| Epha3 | BB292785 | Eph receptor A3 | 2.07 |
| Rnpc2 | BB203348 | Mus musculus 7 days neonate cerebellum cDNA | 2.08 |
| Fmo4 | AF461145 | flavin containing monooxygenase 4 | 2.09 |
| Enc1 | BG976607 | ectodermal-neural cortex 1 | 2.11 |
| | BC019420 | RIKEN cDNA 5730437N04 gene | 2.12 |
| Ddr1 | BB234940 | discoidin domain receptor family, member 1 | 2.13 |
| Gal | NM_01025 | galanin | 2.13 |
| LOC219134 | BC024118 | Hypothetical protein BC024118 | 2.19 |
| Sspn | BC021484 | Sarcospan | 2.26 |
| Asb4 | AV113827 | ankyrin repeat and SOCS box-containing protein 4 | 2.28 |
| Dscr1l1 | AB061524 | Down syndrome critical region gene 1-like 1 | 2.33 |
| Asb4 | AV302111 | ankyrin repeat and SOCS box-containing protein 4 | 2.40 |
| Lfi204 | NM_00832 | interferon activated gene 204 | 2.58 |
| Slc2a3 | M75135 | solute carrier family 2 (facilitated glucose transporter) | 2.64 |
| Mab21l1 | AF228913 | Mab-21-like 1 (C. elegans) | 3.14 |
| Mt1 | NM_01360 | metallothionein 1 | 3.16 |
| Msx2 | NM_01360 | homeo box, msh-like 2 | 3.19 |
| Mal | NM_01076 | myelin and lymphocyte protein, T-cell differentiation | 3.28 |
| 2310010M10Rik | BC006901 | protein | 4.13 |
| Elavl3 | AU067745 | RIKEN cDNA 2310010M10 gene RIKEN cDNA | 4.14 |
| Mt1 | BC027262 | 2600009P04 gene metallothionein 1 | 18.45 |

6. Abnormal Patterning of the Tracheal-Bronchial Cartilage

Malformed tracheal-bronchial cartilaginous rings are observed in Shh^{Δ/Δ} and Shh^{Δ/-} mice. Incomplete tracheal rings (rings 1-4) are observed along the ventral midline. Other abnormalities consist of a reduced number of tracheal-bronchial cartilage rings, incomplete or misaligned tracheal rings, and focal constrictions in tracheal diameter. In Shh^{-/-} mice, the tracheal-bronchial cartilages fail to form and only a small cluster of alcian blue-stained cartilaginous cells is detected in the middle of the presumptive tracheal-esophageal tube. Expression of SHH in the Shh^{rescue} mice does not restore tracheal-bronchial cartilage ring formation or patterning, indicating a local requirement for SHH in the conducting airway.

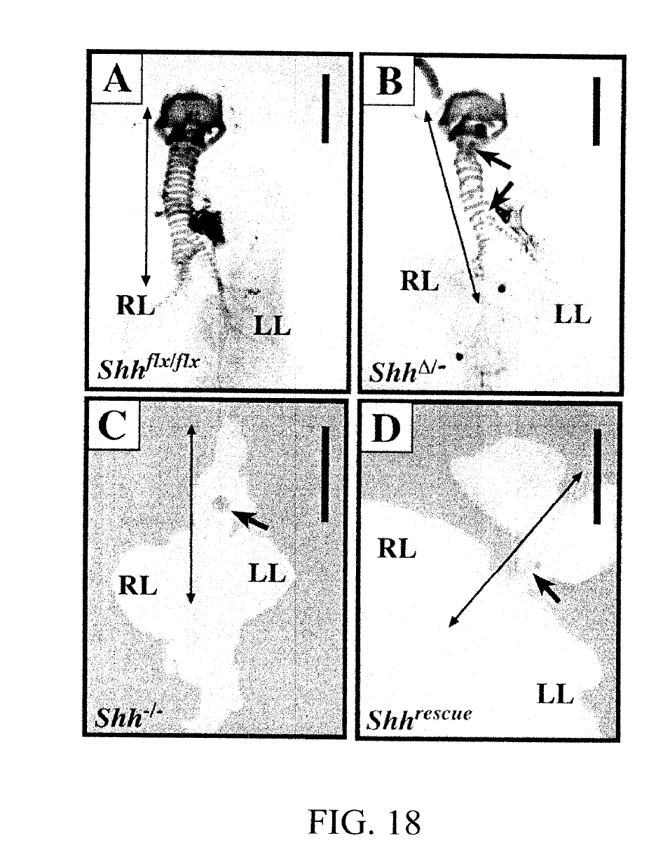

As shown in FIG. 18 (orientation of lungs is indicated by the long arrows: RL=right lung; LL=left lung; scale bar=1 μm; n=3-4 for each genotype), lungs and tracheas removed from Shh^{flx/flx} (see A), Shh^{Δ/-} mice (see B), Shh^{-/-} (see C), and Shh^{rescue} (see D) mice at E18.5 are stained with Alcian blue for detection of cartilage. Tracheal rings in control littermates Shh^{flx/flx} form an almost complete circle and are attached dorsally to a fibrous membrane. Alcian blue staining reveals abnormal tracheal-bronchial cartilage patterning in Shh^{Δ/-} mice wherein tracheal rings 1-4 fail to connect ventrally (see arrows in B). Various rings along the length of the trachea are incomplete or misaligned. The tracheoesophageal fistula observed in Shh^{-/-} and Shh^{rescue} mice contain a cluster of light blue cartilaginous cells (see arrows in C and D, respectively) indicating that loss of tracheal-bronchial cartilage patterning observed in the Shh^{-/-} mice is not restored in the Shh^{rescue} mice.

7. Ptch1 Temporal and Spatial Expression Pattern

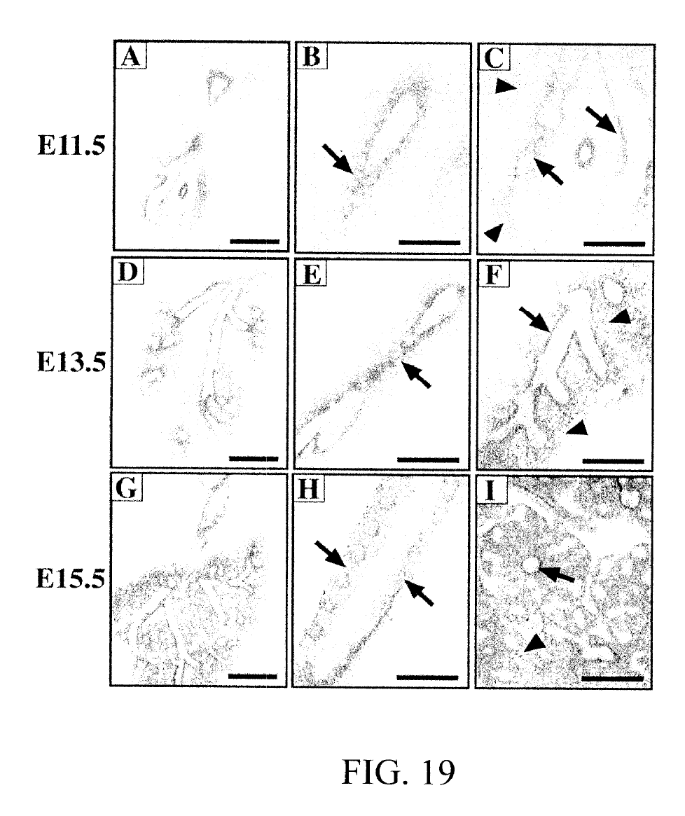

As shown in FIG. 19 (n=3 for each time point; scale bar=200 μm for A, D and G, and 100 μm for B-C, E-F and H-I), in order to assess sites of SHH signaling, expression of Ptch1 is assessed in lung tissue from Ptch1^{LacZ/+} transgenic mice at E11.5 (see A-C), E13.5 (see D-F) and E15.5 (see G-I). β-Galactosidase staining is observed in the mesenchyme at discrete sites surrounding the trachea and bronchi at all of the timepoints studied, being most abundant in the precartilagenous condensations (see arrows in B, E and H). This spatially restricted expression of Ptch1 indicates potential sites of SHH signaling. In the peripheral lung, β-galactosidase staining is detected in the mesenchyme of the peripheral lung (see arrowheads in C, F and I) and along the conducting airways consistent with sites of pulmonary vessel and bronchial smooth muscle formation (see arrows in C, F and I). Sites of Ptch1 gene expression are similar to those detected by in situ hybridization of Ptch1 mRNA at E13.5 (see E-F).

8. Determination of Temporal Requirements for SHH During Lung Morphogenesis

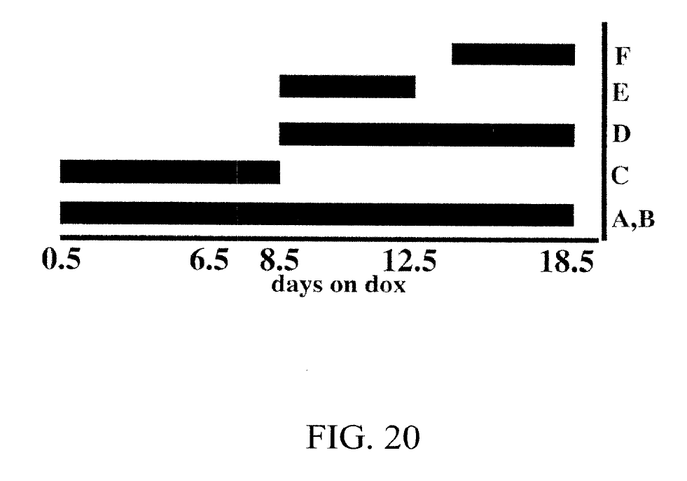

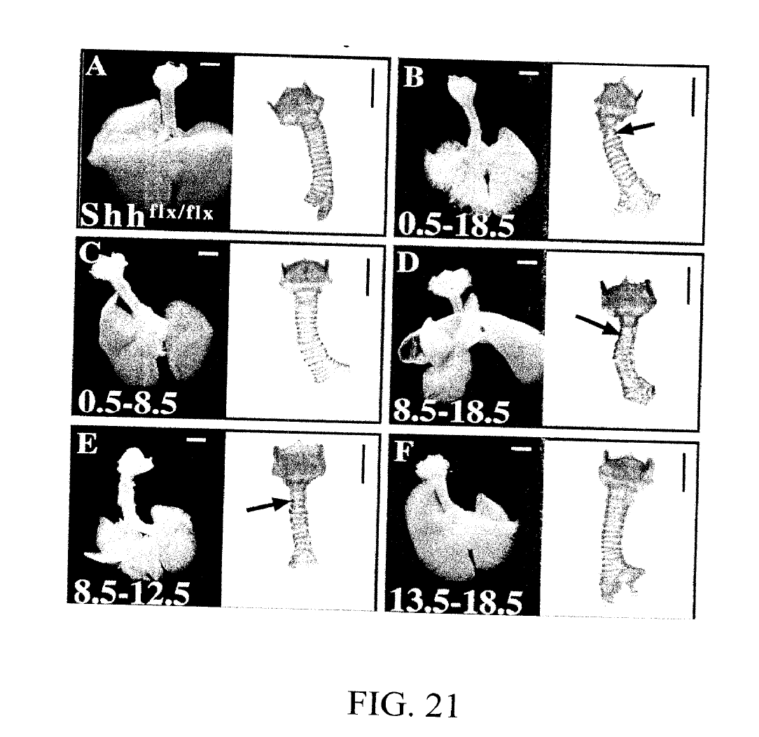

As shown in FIG. 21 (n>5 for each time point; scale bar=1 μm for A-F), deletion of SHH causes the development of severely malformed peripheral lung and conducting airways when doxycycline is administered to Shh^{Δ/Δ} and Shh^{Δ/-} mice from E0.5-18.5 (see B), E8.5-18.5 (see D) and E8.5-12.5 (see E). (Doxcycline treatment times are shown in FIG. 20.) Lungs are harvested at E18.5 for all treatments and analyzed for hypoplasia, presence of lung cysts, peripheral tubule dilation and abnormal tracheal rings (arrows). Shh^{Δ/Δ} and Shh^{Δ/-} mice treated with doxycycline from E13.5 or later result in no observable pulmonary or extrapulmonary abnormalities (see F). Formation of peripheral lung is affected when dams are treated with doxycycline from E0.5-8.5 (see C), E6.5-8.5, E8.5-10.5 or E6.5-E10.5 (not shown). Tracheal abnormalities are not observed in Shh^{Δ/Δ} and Shh^{Δ/-} mice treated with doxycycline before E8.5 (see C), a time during which gene targeting occurs only in intrapulmonary airways (see Perl et al, Transgenic Res., (2002) 11:21-29). Administration of doxycycline for 96 hrs or more, between E8.5 and E12.5 when recombination occurs in epithelial cells of the trachea and main stem bronchi (see Perl et al, Transgenic Res., (2002) 11:21-29), causes severe malformations in the tracheal-bronchial cartilage rings (see E). Therefore, local deletion of Shh in respiratory epithelial cells of the conducting airway causes abnormalities in tracheal-bronchial cartilage patterning.

While specific embodiments of the present invention have been described, it will be apparent to those skilled in the art that various modifications thereto can be made without departing from the spirit and scope of the present invention as defined in the appended claims.

1. A pharmaceutical composition comprising the cartilage formation inducing protein in an amount effective to induce cartilage formation.

2. (canceled)

3. The composition of claim 1 wherein the FGF-18 protein is in combination with the target gene protein.

4-6. (canceled)

7. An expression vector comprising at least one nucleotide sequence encoding at least one cartilage formation inducing protein selected from the group consisting of FGF-18, Shh, β-catenin, and Wnt proteins.

8. The vector of claim 7 wherein the at least one cartilage formation inducing protein is FGF-18 protein.

9-29. (canceled)

30. The pharmaceutical composition of claim 1, wherein the FGF-18 protein is adapted to be administered to an affected area of a patient in an amount effective to induce cartilage formation in the affected area.

31. The pharmaceutical composition of claim 30, wherein the affected area is a conducting airway.

32. The pharmaceutical composition of claim 31, wherein the conducting airway is at least one of the trachea, bronchi, lung and larynx.

33. The pharmaceutical composition of claim 1, further including at least one of a Bone Morphogenetic Protein and a Transforming Growth Factor, the Bone Morphogenetic Protein and the Transforming Growth Factor adapted to be administered to the affected area in an amount effective to enhance cartilage growth and patterning.

34. The expression vector of claim 7, wherein the at least one nucleotide sequence is adapted to be administered to an affected area of a patient in an amount effective to induce cartilage formation in the cells of the affected area.

35. The expression vector of claim 34, wherein the affected area is a conducting airway.

36. The expression vector of claim 35, wherein the conducting airway is at least one of the trachea, bronchi, lung and larynx.

37. A cell culture comprising a first group of cells in a medium capable of sustaining cell growth, the cells having introduced therein an expression vector comprising at least one nucleotide sequence encoding at least one cartilage formation inducing protein selected from the group consisting of FGF-18, Shh, β-catenin, and Wnt proteins.

38. The cell culture of claim 37, wherein the at least one cartilage formation inducing protein is FGF-18.

39. The cell culture of claim 37, further comprising a second group of cells capable of producing cartilage in the presence of the at least one cartilage formation inducing protein, the first group of cells providing in the medium an effective amount of the at least one cartilage formation inducing protein to induce cartilage formation in the second group of cells.

40. The cell culture of claim 39, wherein the at least one cartilage formation inducing protein provided in the medium is FGF-18.

41. The cell culture of claim 39, wherein the at least one cartilage formation inducing protein provided in the medium is FGF-18 in combination with at least one target gene protein selected from the group consisting of the Shh, β-catenin, and Wnt proteins.


SEQUENCE LISTING (14 sequences)

SEQ ID NO: 1 - DNA, 624 bp, House Mouse
atgtattcagcgccctccgcctgcacttgcctgtgtttacactttctactgctgtgcttccaggttcaggtgttggcagccgaggagaatgtggacttccgcatccacgtggagaaccagacgcgggctcgagatgatgtgagtcggaagcagctgcgcttgtaccagctctatagcaggaccagtgggaagcacattcaagttctgggccgtaggatcagtgcccgtggcgaggacggggacaagtatgcccagctcctagtggagacagataccttcgggagtcaagtccggatcaagggcaaggagacagaattctacctgtgtatgaaccgaaaaggcaagctcgtggggaagcctgatggtactagcaaggagtgcgtgttcattgagaaggttctggaaaacaactacacggccctgatgtctgccaagtactctggttggtatgtgggcttcaccaagaaggggcggcctcgcaagggtcccaagacccgcgagaaccagcaagatgtacacttcatgaagcgttaccccaagggacaggccgagctgcagaagcccttcaaatacaccacagtcaccaagcgatcccggcggatccgccccactcaccccggctag

SEQ ID NO: 2 - protein, 207 aa, House Mouse
MYSAPSACTCLCLHFLLLCFQVQVLAAEENVDFRIHVENQTRARDDVSRKQLRLYQLYSRTSGKHIQVLGRRISARGEDGDKYAQLLVETDTFGSQVRIKGKETEFYLCMNRKGKLVGKPDGTSKECVFIEKVLENNYTALMSAKYSGWYVGFTKKGRPRKGPKTRENQQDVHFMKRYPKGQAELQKPFKYTTVTKRSRRIRPTHPG

SEQ ID NO: 3 - DNA, 1406 bp, Human
gtgacgctttcgcgctgcagccgcgcgccccgaccccggagcgctgacccctggccccacgcagctccgcgcccgggccggagagcgcaactcggcttccagacccgccgcgcatgctgtccccggactgagccgggcagccagcctcccacggacgcccggacggccggccggccagcagtgagcgagcttccccgcaccggccaggcgcctcctgcacagcggctgccgccccgcagcccctgcgccagcccggagggcgcagcgctcgggaggagccgcgcggggcgctgatgccgcagggcgcgccgcggagcgccccggagcagcagagtctgcagcagcagcagccggcgaggagggagcagcagcagcggcggcggcggcggcggcggcggcggaggcgcccggtcccggccgcgcggagcggacatgtgcaggctgggctaggagccgccgcctccctcccgcccagcgatgtattcagcgccctccgcctgcacttgcctgtgtttacacttcctgctgctgtgcttccaggtacaggtgctggttgccgaggagaacgtggacttccgcatccacgtggagaaccagacgcgggctcgggacgatgtgagccgtaagcagctgcggctgtaccagctctacagccggaccagtgggaaacacatccaggtcctgggccgcaggatcagtgcccgcggcgaggatggggacaagtatgcccagctcctagtggagacagacaccttcggtagtcaagtccggatcaagggcaaggagacggaattctacctgtgcatgaaccgcaaaggcaagctcgtggggaagcccgatggcaccagcaaggagtgtgtgttcatcgagaaggttctggagaacaactacacggccctgatgtcggctaagtactccggctggtacgtgggcttcaccaagaaggggcggccgcggaagggccccaagacccgggagaaccagcaggacgtgcatttcatgaagcgctaccccaaggggcagccggagcttcagaagcccttcaagtacacgacggtgaccaagaggtcccgtcggatccggcccacacaccctgcctaggccaccccgccgcggccctcaggtcgccctggccacactcacactcccagaaaactgcatcagaggaatatttttacatgaaaaataaggattttattgttgacttgaaacccccgatgacaaaagactcacgcaaagggactgtagtcaacccacaggtgcttgtctctctctaggaacagacaactctaaactcgtccccagaggaggacttgaatgaggaaaccaacactttgagaaaccaaagtcctttttcccaaaggttctgaaaggaaaaaaaaaaaaaaaaaaaaaaaaaaaaaaaa

SEQ ID NO: 4 - protein, 208 aa, Human
MMYSAPSACTCLCLHFLLLCFQVQVLVAEENVDFRIHVENQTRARDDVSRKQLRLYQLYSRTSGKHIQVLGRRISARGEDGDKYAQLLVETDTFGSQVRIKGKETEFYLCMNRKGKLVGKPDGTSKECVFIEKVLENNYTALMSAKYSGWYVGFTKKGRPRKGPKTRENQQDVHFMKRYPKGQPELQKPFKYTTVTKRSRRIRPTHPA

SEQ ID NO: 5 - DNA, 2716 bp, House Mouse
ccttgctaccatttaaaatcaggctctttttgtcttttaattgctgtctcgagacccaactccgatgtgttccgttaccagcgaccggcagcctgccatcgcagccccagtctgggtggggatcggagacaagtcccctgcagcagcggcaggcaaggttatataggaagagaaagagccaggcagcgccagagggaacgaacgagccgagcgaggaagggagagccgagcgcaaggaggagcgcacacgcacacacccgcgcgtacccgctcgcgcacagacagcgcggggacagctcacaagtcctcaggttccgcggacgagatgctgctgctgctggccagatgttttctggtgatccttgcttcctcgctgctggtgtgccccgggctggcctgtgggcccggcagggggtttggaaagaggcggcaccccaaaaagctgacccctttagcctacaagcagtttattcccaacgtagccgagaagaccctaggggccagcggcagatatgaagggaagatcacaagaaactccgaacgatttaaggaactcacccccaattacaaccccgacatcatatttaaggatgaggaaaacacgggagcagaccggctgatgactcagaggtgcaaagacaagttaaatgccttggccatctctgtgatgaaccagtggcctggagtgaagctgcgagtgaccgagggctgggatgaggacggccatcattcagaggagtctctacactatgagggtcgagcagtggacatcaccacgtccgaccgggaccgcagcaagtacggcatgctggctcgcctggctgtggaagcaggtttcgactgggtctactatgaatccaaagctcacatccactgttctgtgaaagcagagaactccgtggcggccaaatccggcggctgtttcccgggatccgccaccgtgcacctggagcagggcggcaccaagctggtgaaggacttacgtcccggagaccgcgtgctggcggctgacgaccagggccggctgctgtacagcgacttcctcaccttcctggaccgcgacgaaggcgccaagaaggtcttctacgtgatcgagacgctggagccgcgcgagcgcctgctgctcaccgccgcgcacctgctcttcgtggcgccgcacaacgactcggggcccacgcccgggccaagcgcgctctttgccagccgcgtgcgccccgggcagcgcgtgtacgtggtggctgaacgcggcggggaccgccggctgctgcccgccgcggtgcacagcgtgacgctgcgagaggaggaggcgggcgcgtacgcgccgctcacggcgcacggcaccattctcatcaaccgggtgctcgcctcgtgctacgctgtcatcgaggagcacagctgggcacaccgggccttcgcgcctttccgcctggcgcacgcgctgctggccgcgctggcacccgcccgcacggacggcgggggcgggggcagcatccctgcagcgcaatctgcaacggaagcgaggggcgcggagccgactgcgggcatccactggtactcgcagctgctctaccacattggcacctggctgttggacagcgagaccatgcatcccttgggaatggcggtcaagtccagctgaagcccgacgggaccgggcaaggggcgggcggggcggggagcgactgcgaaataaggaactgatgggaaagcgcacggaaggagacttttaattataagaataattcataataataataataatgataataataataataataagtagggcagtccaaagtagactataaggaagcaaaaaccccggggagttctgttgttatgtttagtttatatatttttttttgaaatttttcgttattgtcttatatgggttgtttttctcctctcctggctatttatttgtttcgtatgaatagatgttttaaaaatatgaacggaccttcaagagccttaactagtttgtgtcttggataatttattattgtgtgaactgtactcacagtgagggaaagattattttgtgaggccaagcaacctgctgaaagtctatttttctacatgtcccttgtcctgcgtttcagaaggcaaacctccgcattcctctcctgctatgctcctgctttcccgcaagtgtaaactaaaacctgctccatgggggtccacaaattatatttttatacacagaattgtaaattagatttttgagagatcaatacctaactgaatgacatttcattttttgaaagtgtaaaatatgaaaatatattattttaatttaactattttccaatgtaatagccgtcttctgtactgccttcttggtttgtatttgctttgtaaccgccactttgtcatgttcttggaaaccaagactgttaacgcacacatatacacttttttttttgacagactggaagaactctgttatttttaacttcaaagaatttattagaaaataatattttttaaaagtgcacctagcagcgagcccacgaggatggagcctgtagtttgtacagagaaaaacaaggatgtttttgcattaataaactgagaagtaactgctgtaaatttactaaaatgtatttttgaatattttgtaatagttttatagaaataaagcgtgccacacacaaaaaaaaaaaaaaaaaaaaaaaaaaaaaaaaaaaaaaaaaaaaaaaaaaaaaaaaaaaaa

SEQ ID NO: 6 - protein, 437 aa, House Mouse
MLLLLARCFLVILASSLLVCPGLACGPGRGFGKRRHPKKLTPLAYLQFIPNVAEKTLGASGRYEGKITRNSERFKELTPNYNPDIIFKDEENTGADRLMTQRCKDKLNALAISVMNQWPGVKLRVTEGWDEDGHHSEESLHYEGRAVDITTSDRDRSKYGMLARLAVEAGFDWVYYGSKAHIHCSVKAENSVAAKSGGCFPGSATVHLEQGGTKLVKDLRPGDRVLAADDQGRLLYSDFLTFLDRDEGAKKVFYVIGTLEPREPLLLTAAHLLFVAPHNDSGPTPGPSALFASRVRPGQRVYVVAERGGDRRLLPAAVHSVTLREEEAGAYAPLTAHGTILINRVLASCYAVIEEHSWAHRAFAPFRLAHALLAALAPARTDGGGGGSIPAAQSATEARGAEPTAGIHWYSQLLYHIGTWLLDSETMHPLGMAVKSS

SEQ ID NO: 7 - DNA, 3565 bp, House Mouse
gcggcgccatcttaagccctcgctcggtggcggccgcgtcagctcgtgtcctgtgaagcccgcggcccggggaggcggagacggagcacggtgggcgccgagccgtcagtgcaggaggccgaggccgagcgggcggccgcgagtgagcagcgcgcgggcctgagggtacctgaagctcagcgcacagctgctgtgacaccgctgcgtggacaatggctactcaagctgacctgatggagttggacatggccatggagccggacagaaaagctgctgtcagccactggcagcagcagtcttacttggattctggaatccattctggtgccaccaccacagctccttccctgagtggcaagggcaaccctgaggaagaagatgttgacacctcccaagtcctttatgaatgggagcaaggcttttcccagtccttcacgcaagagcaagtagctgatattgacgggcagtatgcaatgactagggctcagagggtccgagctgccatgttccctgagacgctagatgagggcatgcagatcccatccacgcagtttgacgctgctcatcccactaatgtccagcgcttggctgaaccatcacagatgttgaaacatgcagttgtcaatttgattaactatcaggatgacgcggaacttgccacacgtgcaattcctgagctgacaaaactgctaaacgatgaggaccaggtggtagttaataaagctgctgttatggtccatcagctttccaaaaaggaagcttccagacatgccatcatgcgctcccctcagatggtgtctgccattgtacgcaccatgcagaatacaaatgatgtagagacagctcgttgtactgctgggactctgcacaacctttctcaccaccgcgagggcttgctggccatctttaagtctggtggcatcccagcgctggtgaaaatgcttgggtcaccagtggattctgtactgttctacgccatcacgacactgcataatctcctgctccatcaggaaggagctaaaatggcagtgcgcctagctggtggactgcagaaaatggttgctttgctcaacaaaacaaacgtgaaattcttggctattacaacagactgccttcagatcttagcttatggcaatcaagagagcaagctcatcattctggccagtggtggaccccaagccttagtaaacataatgaggacctacacttatgagaagcttctgtggaccacaagcagagtgctgaaggtgctgtctgtctgctctagcaacaagccggccattgtagaagctggtgggatgcaggcactggggcttcatctgacagacccaagtcagcgacttgttcaaaactgtctttggactctcagaaacctttcagatgcagcgactaagcaggaagggatggaaggcctccttgggactctagtgcagcttctgggttccgatgatataaatgtggtcacctgtgcagctggaattctctctaacctcacttgcaataattacaaaaacaagatgatggtgtgccaagtgggtggcatagaggctcttgtacgcaccgtccttcgtgctggtgacagggaagacatcactgagcctgccatctgtgctcttcgtcatctgaccagccggcatcaggaagccgagatggcccagaatgccgttcgccttcattatggactgcctgttgtggttaaactcctgcacccaccatcccactggcctctgataaaggcaactgttggattgattcgaaaccttgccctttgcccagcaaatcatgcgcctttgcgggaacagggtgctattccacgactagttcagctgcttgtacgagcacatcaggacacccaacggcgcacctccatgggtggaacgcagcagcagtttgtggagggcgtgcgcatggaggagatagtagaagggtgtactggagctctccacatccttgctcgggacgttcacaaccggattgtaatccgaggactcaataccattccattgtttgtgcagttgctttattctcccattgaaaatatccaaagagtagctgcaggggtcctctgtgaacttgctcaggacaaggaggctgcagaggccattgaagctgagggagccacagctcccctgacagagttactccactccaggaatgaaggcgtggcaacatacgcagctgctgtcctattccgaatgtctgaggacaagccacaggattacaagaagcggctttcagtcgagctgaccagttccctcttcaggacagagccaatggcttggaatgagactgcagatcttggactggacattggtgcccagggagaagcccttggatatcgccaggatgatcccagctaccgttcttttcactctggtggatacggccaggatgccttggggatggaccctatgatggagcatgagatgggtggccaccaccctggtgctgactatccagttgatgggctgcctgatctgggacacgcccaggacctcatggatgggctgcccccaggtgatagcaatcagctggcctggtttgatactgacctgtaaatcgtcctttaggtaagaaagcttataaaagccagtgtgggtgaatactttactctgcctgcagaactccagaaagacttggtagggtgggaatggttttaggcctgtttgtaaatctgccaccaaacagatacataccttggaaggagatgttcatgtgtggaagtttctcacgttgatgtttttgccacagcttttgcagcgttatactcagatgagtaacatttgctgttttcaacattaatagcagcctttctctctatacagctgtagtgtctgaacgtgcattgtgattggcctgtagagttgctgagagggctcgaggggtgggctggtatctcagaaagtgcctgacacactaaccaagctgagtttcctatgggaacagtcgaagtacgctttttgttctggtcctttttggtcgaggagtaacaatacaaatggatttggggagtgactcacgcagtgaagaatgcacacgaatggatcacaagatggcgttatcaaaccctagccttgcttgttctttgttttaatatctgtagtggtgctgactttgcttgcttttattttttgcagtaactgttagtttttaagtagtgttatgttctagtgaacctgctacagcaatttctgatttctaagaaccgagtaatggtgtagaacactaattcataatcacgctaattgtaatctggagacgtgtaacattgtgtagccttttgtataaatagacagatagaaatggtccgattagtttcctttttaatatgcttaaaataagcaggtggatctatttcatgtttttgaacaaaaactttatcggggatacgtgcggtagggtaaatcagtaagaggtgttatttgagccttgttttggacagtataccagttgccttttatcccaaagttgttgtaacctgctgtgatacaatgcttcaacagatgcggttatagaaatggttcagaattaaacttttaattcattcaaaaaaaaaaaaaaaaaa

SEQ ID NO: 8 - protein, 781 aa, House Mouse
MATQADLMELDMAMEPDRKAAVSHWQQQSYLDSGIHSGATTTAPSLSGKGNPEEEDVDTSQVLYEWEQGFSQSFTQQQVADIDGQYAMTRAQRVRAAMFPETLDEGMQIPSTQFDAAHPTNVQRLAEPSQMLKHAVVDLIDYQDDAELATRAIPELTKLLNDEDQVVVNKAAVMVHQLSKKEASRHAIMRSPQMVSAIVRTMQNTNDVETARCTAGTLHNLSHHREGLLAIFKSGGIPALVKMLGSPVDSVLFYAITTLHNLLLHQEGAKMAVRLAGGLQKMVALLNKTNVKFLAITTDCLQILAYGNQESKLIILASGGPQALVNIMRTYTYEKLLWTTSRVLKVLSVCSSKNPAIVEAGGMQALGLHLTDPSQRSVQNCLWTLRNSLDAATKQEGMEGLLGTLVQLLGSDDINVVTCAAGILSNLTCNNYKNKMMVCQVGGIEALVRTVLRAGDREDETEPAICALRHLTSRHQEAEMAQNAVRLHYGLPVVVKLLHPPSHWPLIKATVGLIRNLALCPANHAPLREQGAIPRLVQLLVRAHQDTQRRTSMGGTQQQFVEGVRMEEIVEGCTGALHILARDVHNRIVIRGLNTIPLFVQLLYSPIENIQRVAAGVLCELAQDKEAAEAIEAEGATAPLTELLHSRNEGVATYAAAVLFRMSEDKPQDYKKRLSVELTSSLFRTEPMAWNETADLGLDIGAQGEALGYRQDDPSYRSFHSGGYGQDALGMDPMMEHEMGGHHPGADYPVDGLPDLGHAQDLMDGLPPGDSNQLAWFDTDL

SEQ ID NO: 9 - DNA, 3971 bp, House Mouse
tttgctcagaccggcaagagccacagcttcgctcgccactcattgtctgtggccctgaccagtgcgccctggtgcttttagtgccgcccgggcccggaggggcagcctcttctcactgcagtcagcgccgcaactataagaggcctataagaggcggtgcctcccgcagtggctgcttcagcccagcagccaggacagcgaaccatgctgcctgcggcccgcctccagacttattagagccagcctgggaactcgcatcactgccctcaccgctgtgtccagtcccaccgtcgcggacagcaaccacagtcgtcagaaccgcagcacagaaccagcaaggccaggcaggccatggggctctgggcgctgctgcccagctgggtttctactacgttgctactggcactgaccgctctgcccgcagccctggctgccaacagtagtggccgatggtggtaagtgagctagtacggggtccgccacttgtcctggggcaaagagccaggcacgggccttacccagctcccacgctgtggggatcaccaacctacagacccccctcgtgcattgtgacttcacatccagggtgctcacacctagaactagctctgctgaagtggggcacatcattggcatgcagaagcccagatacaccaggctcagagaccattcccatttaatacgaccccgtttctgctgagcaacaggtcccaacctcgctgtggtgggtgctcaggtgtcccttaggtcttgaaccaaaaaaaaaaaaaaaaaaaaaaaaaaaccagatattagctttgaggtgagggagtggaattcctaagtttttcaaggtgggcaaggctgcaggtggggtttctcctcgggggctgacttgaagaaaggaagagctaaggtagccatgccttttctgtccactcactagactctggagctcagggccaggcaaggatagggtggtacagcctgtatggttaggatgcaggtcccctcccctggactgaacccttatgcatcccgccaggggcatcgtgaacatagcctcctccacgaacctgttgacggattccaagagtctgcagctggtgctcgagcccagtctgcagctgctgagccgcaagcagcggcgactgatccgacagaacccggggatcctgcacagcgtgagtggagggctccagagcgctgtgcgagagtgcaaatggcaattccgaaaccgccgctggaactgccccactgctccggggccccacctcttcggcaagatcgtcaaccgaggtgggtgcccaggaaagcgacgcttccgggattaagggaaaagcagggtcatctccagggcataggcgggcgaaggcagggaagacatcccagggttatatgtgatcaaactgagaatcgcctggtgccggcagttaccgtaggtcagcaccagattctttctagccttgcgttgtgagcatgatctttaacgttgctggccactggcccacagaaagggaattccggatcgtgggcgctgggcgacagctgtttttccctagccttcctcaaaggtacctgggaagctgatctctgagggctagctagggttgtgcttcgcacccagcaaagtttgcactgccaatactagtagcgatcttggctatgcagatttgttctacttgggaatctccccttggagctgctctgctagggctctggagtctcagtaaagcttagagaggagggcattccatgcttcgcacacatgactccaaggatgttggactgtagggtaccaagtcttccaaacagggtgctgagttggccccacgccttctctcaactgatgcggggtcgcttcacccacaggctgccgagaaacagcgttcatcttcgcaatcacctccgccggggtcacacattccgtggcgcgctcctgctccgaaggctccatcgagtcctgcacctgcgactaccggcggcgcggccctgggggccccgactggcactgggggggctgcagtgacaacatcgattttggtcgcctctttggccgagagttcgtggactccggggagaaggggcgggacctacgcttcctcatgaaccttcacaacaacgaggcagggcgaacggtacgtcggtgtgtccggaaccaatggcaggggagatgtaagacaggtgcacggggacagaggcacagggaggggcttcccgagagagtgggactctaggagggaagacagagaagaggtggtggttgagggcaaagaggttcctgagctgatgacagaacagaagagattagcaggctatcaacacgtgggatgtattgagatggctccatggcacacttttgaaagataaaagtgacttgctggcgtggagcagagtctggccgaatgtccctatctcagcgggccattttgcacttcctctctcccgagcttagtcacacctggaccttggctgaagtttccacagcatcgacgtgacccgggtggggtgggggtggggaagtatgggtggtggttcgtgggatgttggctttgaccttttcttccctcctcccctcgtcccctcctcccccagaccgtgttctctgagatgcgccaagagtgcaaatgccacgggatgtccggctcctgcacggtgcgcacgtgttggatgcggctgcccacgctgcgcgctgtgggcgacgtgctgcgcgaccgcttcgacggcgcctcccgcgtcctttacggcaaccgaggcagcaaccgcgcctcgcgggcggagctgctgcgcctggagcccgaagaccccgcgcacaagcctccctcccctcacgacctcgtctacttcgagaaatcgcccaacttctgcacgtacagtggccgcctgggcacagctggcacagctggacgagcttgcaacagctcgtctcccgcgctggacggctgtgagctgctgtgctgtggccgaggccaccgcacgcgcacgcagcgcgtcacggagcgctgcaactgcaccttccactggtgctgccacgtcagctgccgcaactgcacgcacacgcgcgttctgcacgagtgtctatgaggtgccgcgcctccgggaacgggaacgctctcttccagttctcagacacactcgctggtcctgatgtttgcccaccctaccgcgtccagccacagtcccagggttcatagcgatccatctctcccacctcctacctggggactcctgaaaccacttgcctgagtcggctcgaacccttttgccatcctgagggccctgacccagcctacctccctccctctttgagggagactccttttgcactgccccccaatttggccagagggtgagagaaagattcttcttctggggtgggggtggggaggtcaactcttgaaggtgttgcggttcctgatgtattttgcgctgtgacctctttgggtattatcacctttccttgtctctcgggtccctataggtcccttgagttctctaaccagcacctctgggcttcaaggcctttcccctcccacctgtagctgaagagtttccgagttgaaagggcacggaaagctaagtgggaaaggaggttgctggacccagcagcaaaaccctacattctccttgtctctgcctcggagccattgaacagctgtgaaccatgcctccctcagcctcctcccaccccttcctgtcctgcctcctcatcactgtgtaaataatttgcaccgaaatgtggccgcagagccacgcgttcggttatgtaaataaaactatttattgtgctgggttccagcctgggttgcagagaccaccctcaccccacctcactgctcctctgttctgctcgccagtccttttgttatccgaccttttttctcttttacccagcttctcataggcgcccttgcccaccggatcagtatttccttccactgtagctattagtggctcctcgcccccaccaatgtagtatcttcctctgagga

SEQ ID NO: 10 - protein, 370 aa, House Mouse
MGLWALLPSWVSTTLLLALTALPAALAANSSGRWWGIVNIASSTNLLTDSKSLQLVLEPSLQLLSRKQRRLIRQNPGILHSVSGGLQSAVRECKWQFRNRRWNCPTAPGPHLFGKIVNRGCRETAFIFAITSAGVTHSVARSCSAGSIESCTCDYRRRGPGGPDWHWGGCSDNIDFGRLFGREFVDSGEKGRDLRFLMNLHNNEAGRTTVFSEMRQECKCHGMSGSCTVRTCWMRLPTLRAVGDVLRDRFDGASRVLYGNRGSNRASRAELLRLEPEDPAHKPPSPHDLVYFEKSPNFCTYSGRLGTAGTAGRACNSSSPALDGCELLCCGRGHRTRTQRVTERCNCTFHWCCHVSCRNCTHTRVLHECL

SEQ ID NO: 11 - DNA, 1669 bp, House Mouse
ccgccgcgccctcctcgcccgggatgggcccccccgccgccaccgccgccggagccctagtctccgggccgccgcctcggtcgccgcgtttgccctgaagcccggtgcccgcgcgccccggctcaccccgcagcttcactccccacccccagccgcctccccggccagactgcggtagagctctcaggatgctgccgccggtgccctcccgcctcggactgctgctgctgctcttgtgccccgcgcacgtcgatggactgtggtgggccgtgggcagccccttggtcatggatcctaccagcatctgcaggaaggccaggcggctggcaggaagacaggccgagctgtgccaggcggagccggaagtagtggcagagcttgcccgaggcgcaagactgggggttcgagaatgtcagttccagttccgtttccgacgctggaactgctccagccacagcaaggcctttgggcgcgtcctgcagcaggacatccgagagacagctttcgtgtttgcaatcaccgcagctggtgccagccacgcggtcactcaagcctgttccatgggagagctcctacagtgtggttgtcaggcaccccgcgggcgggcaccgcctaggccctccggccttctgggcactcctggacctccaggaccaactggctctccagatgctagcgcagcctgggagtggggaggctgcggagacgatgtggacttcggggatgagaagtcaagactctttatggatgcgcagcacaagcggggccgtggagatatccgtgcattggtgcaactgcacaacaacgaggcgggcaggctggcggtgcggagtcacacgcgcaccgagtgtaagtgccatgggctttcgggttcctgcgctctgcgcacctgctggcagaagctgcctccgttccgcgaggtgggcgcacggctgctggagcgcttccacggcgcctcgcgcgtcatgggcaccaacgacggcaaagctctgctgcctgcggtccgcacactcaagcctcccggacgagcggatctcctctacgcagccgattcacccgacttctgcgcccccaaccggcgcacgggttcgccgggcacgcgcggacgcgcctgcaacagcagtgccccggacctcagcggctgcgacctgttgtgctgcggtcgcgggcaccgccaggagagcgtacagctcgaggagaactgtctgtgccgcttccactggtgctgcgtggtgcaatgccaccgctgccgggtgcgcaaggaactcagcctgtgcctctgacccgtcgcctgcctcggaactgctggcagccacctctgggccatctacaggactattagattccagcagggggcgctgtctgagtccagcagctccctaggaaaagtacctatccaggccttgggaaattacaggggccagccaggaacttggggtttacaccagcccacgaaagcccgggggaacatacccctccagcattcccctgaaaggccctttgctagttcctgcaggagatcactccccttggccccccagatggaaataagaaagccagactctgccctctggaaataatattcctcagaattactgggatggatgggtgagtttagtatcaataaagacatttaaatccacaaaaaaaaaaaaaaaaaaaaaaaaaa

SEQ ID NO: 12 - protein, 364 aa, House Mouse
MLPPVPSRLGLLLLLLCPAHVDGLWWAVGSPLVMDPTSICRKARRLAGRQAELCQAEPEVVAELARGARLGVRECQFQFRFRRWNCSSHSKAFGRVLQQDIRETAFVFAITAAGASHAVTQACSMGELLQCGCQAPRGRAPPRPSGLLGTPGPPGPTGSPDASAAWEWGGCGDDVDFGDEKSRLFMDAQHKRGRGDIRALVQLHNNEAGRLAVRSHTRTECKCHGLSGSCALRTCWQKLPPFREVGARLLERFHGASRVMGTNDGKALLPAVRTLKPPGRADLLYAADSPDFCAPNRRTGSPGTRGRACNSSAPDLSGCDLLCCGRGHRQESVQLEENCLCRFHWCCVVQCHRCRVRKELSLCL

SEQ ID NO: 13 - DNA, 3154 bp, House Mouse
cgcccgcctcccgagccgaagcgccggctgagcgtggtcctaccgcagctccctggctcctgcccggcccctgcccacccgcgcgtcccctccggccgcagctgtctatggcgcagcccccctccctggatcatgcacagaaactttcgaaagtggatcttttacgtgtttctctgctttggcgtcctctacgtgaagctcggagcattgtcatccgtggtggccctggtagccaacatcatctgcaacaagattcctggcctggccccacggcagcgtgccatctgccagagccgacccgatgccatcattgtgatcggggagggggcgcagatgggcatcgacgagtgccagcaccagttccgattcggccgctggaactgctccgccctgggcgagaagaccgtcttcgggcaagaactccgagtagggagtcgagaggctgccttcacctatgccatcacggcggcgggcgtggcgcatgctgtcaccgctgcctgcagccagggcaatctgagcaattgtggctgtgaccgggagaagcaaggctactacaaccaggcggaaggctggaagtgggggggctgctcagcggacgtccgctacggcatcgacttttctcgtcgctttgtggatgcccgtgagatcaaaaagaacgccaggcgcctcatgaaccttcacaacaatgaggcgggcagaaaggttctggaggaccgcatgaagctggaatgtaagtgtcacggtgtgtcaggctcctgtaccaccaaaacttgctggaccacgctacctaagttccgcgaggtgggccacctgctcaaggagaagtacaacgcagcggtgcaggtggaggtggtgcgagccagccgcctgcgccagcccaccttcctgcgcatcaagcagctacgcagctaccagaagcctatggagacggacctggtgtacatcgagaagtcgcccaactactgcgaggaggacgcggccacgggcagcgtgggcacgcagggccgtctgtgcaaccgcacctcgccgggggccgacggctgtgacaccatgtgctgcggccgcggctacaacacgcaccagtacaccaaggtgtggcagtgtaactgcaaattccactggtgttgcttcgtcaagtgcaacacgtgcagcgagcgcaccgaggtcttcacctgcaagtgaggctcccgcgcaggcgcgctcggcccctgccgaccctgcggccctcgccattattttgcacatccttctttgcttctggagctgccagctgcaggcacaggagggtggggatagaggtggggagctcgagatactccaggctccttcctactcgctctgtccccgcccagcatccaaggtcaacgcaatggtggtctggtacccaatggagacaaatccctttacttctctttgggaaagtgaaccacaaagggaccatgagactctgagggtcacctccctgcctgtgactggacacagaaaggccacacccaccagtcacactcaaaacggtttcctgggctgtttcctgccggccctgggcagtgtggatggatgttgacaaaattatttatgttttcttagcatcagatgaggactcagtactaacgactgggtagccagacctaaccctatttgaggacacccttccctcactcctcccggcccctccctgcagggtcctctgctccttgcagaactcgaggatgtcagaattggcacggaagctggctggtggggggactccttatcagcaccttgggaggggcttggtggccctacaaggcctgagatggccgcagaggacagccaatcttccattccatttggagactgtcatgcaaatcaaatgtcccttgtgtcaggctccaggcatgcctcgtcctctccctggtccttcaccctcccagcctgctgccaacctccacctccagtttacaaattctcttctcctctggagccaacctgacacccaggactgccccacaggttcaggagaggtcagggacagttgccccacatgacagatggacagagggcaatctgaagatttactggagaccccacggctctgtgaaataaatatactgacacagccccatccagcccaactctggaagttgccagggtgatgggaggctgcacccccttttcagtaccttgggttttgtccttcttctgtgatcctgatgccagagaactgacatccagaatttagggatgtattggtcaggccccctgcctagtgtccactgatacctgcttcagggtccttatattatgaggacatgggaccctcaaacaggggtccgtgggaagcttaatgtcccatttcctcaggcccttccagatggggacagaagaactcaggcctgggcatatcccaccctttcctccacaacacatggcagggtaagaaactgccagggctgataatacaactgcccacagcctaccccacactaaggtgtttcatagcagaagtccatggaaatgtggggtttggtggccaccaagccaggtggcctggacattgacctggggaaggtgacccttgtttgcccttgccttgcatccagctgtgtgtccctatcatgtcaggatgttccaagcctctgggccactggaaatgtcccaccctgatcctggccccatctcctcaccccaagtcctgggatacccacgtccgtcgcccagtgtcccctgtgaggagcctggttaacttatattgttatatagcgtcccctgtctgtcatgtctcttaagttattgtgacctacactgggtaccggaggggatgggggatggcttcagctgctgtcccccaagccaggctcctccttctgcttgaaacagaccctcgggggcccctgatgccaccgaggcaattcgcactgtccctgggctgccaggcacctgcgcctgcactcggtcagccgcagaccttgccttgggggagagaggtggttagtggacccaggcagggcactggctgtcccaatgctgtgtgctggggtggaggtggccgggcaccacatgtccttgaagtgccctacttctgatgggctgtgttcctgcctcctctggaggggagcacttagccccaataaaagctggaatcagaaaaaaaaaaaaaaaaaaaaaaaaaaaa

SEQ ID NO: 14 - protein, 349 aa, House Mouse
MHRNFRKWIFYVFLCFGVLYVKLGALSSVVALVANIICNKIPGLAPRQRAICQSRPDAIIVIGEGAQMGIDECQHQFRFGRWNCSALGEKTVFGQELRVGSREAAFTYAITAAGVAHAVTAACSQGNLSNCGCDREKQGYYNQAEGWKWGGCSADVRYGIDFSRRFVDAREIKKNARRLMNLHNNEAGRKVLEDRMKLECKCHGVSGSCTTKTCWTTLPKFREVGHLLKGKYNAAVQVEVVRASRLRQPTFLRIKQLRSYQKPMETDLVYIEKSPNYCEEDAATGSVGTQGRLCNRTSPGADGCDTMCCGRGYNTHQYTKVWQCNCKFHWCCFVKCNTCSERTEVFTCK